(19) United States
(12) Patent Application Publication
(10) Pub. No.: US 2007/0232687 A1
(43) Pub. Date: Oct. 4, 2007

(54) Agent for Skin External Use Containing Tocopherol Derivative, Ascorbic Acid Derivative and Surface Active Agent Having Lipopeptide Structure

(76) Inventors: Eiko Kato, Chiba (JP); Toshi Tsuzuki, Chiba (JP); Eiji Ogata, Chiba (JP)

Correspondence Address: SUGHRUE MION, PLLC, 2100 PENNSYLVANIA AVENUE, N.W., SUITE 800, WASHINGTON, DC, 20037, US

(21) Appl. No.: 11/587,809

(22) PCT Filed: Apr. 19, 2005
(86) PCT No.: PCT/JP05/07758

§ 371 Date: Oct. 26, 2006

Related U.S. Application Data
(60) Provisional application No. 60/567,528, filed on May 4, 2004.

(30) Foreign Application Priority Data
Apr. 26, 2004 (JP) 2004-130205

(51) Int. Cl. A61K 31/355

(52) U.S. Cl. 514/458

(57) ABSTRACT

The agent for skin external use of the present invention contains (A) tocopherol glycine esters, (B) ascorbic acid-2-phosphoric acid esters and a specific amount of (C) an anionic surface active agent having a lipopeptide structure. According to the agent for skin external use (including a cosmetic) of the present invention, it is possible to enhance stability of the tocopherol glycine esters in spite that the tocopherol glycine esters and the ascorbic acids are contained simultaneously, so that occurrence of coloring (including turbidity) and/or precipitation with time can be prevented. Therefore, efficacy and effects of the agent or the cosmetic can be well exerted over a long period of time.

### REFERENCE TO RELATED APPLICATIONS

This application is an application filed under 35 U.S.C. §111(a) claiming benefit pursuant to 35 U.S.C. §119(e) of the filing dates of Provisional Application No. 60/567,528 filed on May 4, 2004 pursuant to 35 U.S.C. §111(b).

### FIELD OF THE INVENTION

The present invention relates to an agent for skin external use which comprises a tocopherol derivative, an ascorbic acid derivative and a surface active agent having a lipopeptide structure and shows excellent stability and solubility.

### BACKGROUND OF THE INVENTION

It is known that tocopherol (α-tocopherol, β-tocopherol, γ-tocopherol, δ-tocophrerol or the like) known as vitamin E, and its derivatives such as tocopheryl acetate and tocopheryl nicotinate (hereinafter referred to “tocopherols” inclusively) exhibit efficacy and effects in anti-oxidation action, biomembrane stabilizing action, immunity activation action and blood circulation acceleration action, etc., and they have been added to medicines, cosmetics, feeds and the like for a long time.

Most of the tocopherols, however, are oil-soluble, and it was difficult to homogeneously disperse themselves directly in an aqueous solution or emulsion. Therefore, in the preparation of medicines or cosmetics containing the tocopherols in a dissolved or emulsified state, they are generally dispersed in an aqueous solution or emulsion using nonionic surface active agents. However, some nonionic surface active agents are highly irritant or become causative agents of environmental pollution. Therefore, use of such nonionic surface active agents is considered to be undesirable from the viewpoint of safety or the like, and the improvement thereof has been desired.

Further, since tocopherol is easily oxidized and unstable when used alone, it is often used in the form of a derivative of an organic acid ester such as acetate, nicotinate or succinate. For allowing such a derivative to exhibit physiological activity as tocopherol in the living organism, the ester linkage portion thereof needs to be hydrolyzed by an enzyme such as esterase to convert the derivative into an active tocopherol. The conversion rate, however, is not always satisfactory, and the effect of enhancing the concentration in a biological tissue is low.

In connection therewith, the present inventors have found that in order to improve solubility and emulsifiability of the tocopherols in agents for skin external use and to efficiently convert them to active tocopherol in the skin tissues, it is effective to use tocopherol glycine ester and/or its salt or a tocopherol glycine ester derivative and/or its salt (all of them being referred to as “tocopherol glycine esters” hereinafter), and they have already reported an agent for skin external use that contains such a component (see patent document 1 and patent document 2).

By the way, as substances exhibiting anti-oxidation action in the living organism, there are known ascorbic acid known as vitamin C and its derivatives such as ascorbic acid-2-phosphoric acid ester and/or its salt and ascorbic acid-2-glucoside (referred to as “ascorbic acids” inclusively hereinafter) in addition to the above-mentioned tocopherols, and also the ascorbic acids have been added medicines, cosmetics, feeds and the like for a long time.

The ascorbic acids participate not only in capture of water-soluble radicals but also in regeneration of fat-soluble radical capture type oxides such as tocopherols, and therefore, they are expected to more effectively exhibit the anti-oxidation action by formulating both of the tocopherols and the ascorbic acids at the same time and administering them to the living organism.

However, when an agent containing the tocopherol glycine esters and the ascorbic acids simultaneously is intended to be prepared, coloring (including turbidity) or precipitation takes place with time to impair external appearance, and moreover, there is a problem that the stability of the tocopherol glycine esters in an agent is lowered. Accordingly, the development of an agent for skin external use which contains them in a stable state has been desired.

As for stable cosmetic formulations simultaneously containing tocopherols and ascorbic acids, such an embodiment that the agent further contains an ectoin derivative in addition to the tocopherols and ascorbic acids is disclosed in a patent document 3. Further, the embodiment that the agent further contains a non-aqueous ointment base, a polyhydric alcohol and a nonionic surface active agent in addition to the tocopherols and ascorbic acids is disclosed in a patent document 4. In any of these documents, however, there is no description of the tocopherol glycine esters.

Patent document 1: Japanese patent Laid-Open Publication No. 2277/2004

Patent document 2: Japanese patent Laid-Open Publication No. 2278/2004

Patent document 3: National Publication of International Patent No. 522367/2002

Patent document 4: Japanese patent Laid-Open Publication No. 231628/2003

### DISCLOSURE OF THE INVENTION

It is an object of the present invention to provide an agent for skin external use (including a cosmetic), which agent contains at least the tocopherol glycine esters and the ascorbic acids simultaneously and has been improved in lowering of stability with time.

The present inventors have earnestly studied to solve the above problems, and as a result, they have found that an agent for skin external use, which further contains a specific amount of an anionic surface active agent having a lipopeptide structure represented by the following formula (3) in addition to the tocopherol glycine esters and the ascorbic acids, can be improved in lowering of stability with time.

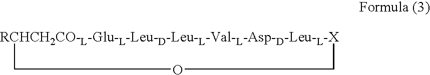

Based on the finding, the present invention has been accomplished.

That is to say, the present invention relates to the following matters.

(1) An agent for skin external use, comprising (A) at least one substance selected from the group consisting of tocopherol glycine ester, a tocopherol glycine ester derivative and a salt thereof, (B) at least one substance selected from the group consisting of ascorbic acid-2-phosphoric acid ester, an ascorbic acid-2-phosphoric acid ester derivative and a salt thereof, and (C) an anionic surface active agent having a lipopeptide structure, wherein the anionic surface active agent (C) having a lipopeptide structure is contained in an amount of 0.03 to 20% by mass.

(2) The agent for skin external use as stated in (1), wherein the component (A) has an α-tocopherol skeleton, γ-tocopherol skeleton or δ-tocopherol skeleton structure.

(3) The agent for skin external use as stated in (1), wherein the component (A) has a dl-α-tocopherol skeleton, d-α-tocopherol skeleton, d-γ-tocopherol skeleton or d-δ-tocopherol skeleton structure.

(4) The agent for skin external use as stated in any one of (1) to (3) wherein the component (A) is an organic acid salt of a tocopherol glycine ester.

(5) The agent for skin external use as stated in any one of (1) to (3) wherein the component (A) is an inorganic acid salt of a tocopherol glycine ester.

(6) The agent for skin external use as stated in (5) wherein the inorganic acid salt of a tocopherol glycine ester is a hydrochloride.

(7) The agent for skin external use as stated in any one of (1) to (3) wherein the component (A) is an organic acid salt of a tocophrol glycine ester derivative.

(8) The agent for skin external use as stated in any one of claims 1 to 3 wherein the component (A) is an inorganic acid salt of a tocophrol glycine ester derivative.

(9) The agent for skin external use as stated in (8) wherein the inorganic acid salt of a tocopherol glycine ester derivative is a hydrochloride.

(10) The agent for skin external use as stated in any one of (1) to (3) and (7) to (9), wherein the tocopherol glycine ester derivative is a compound represented by the formula (1): 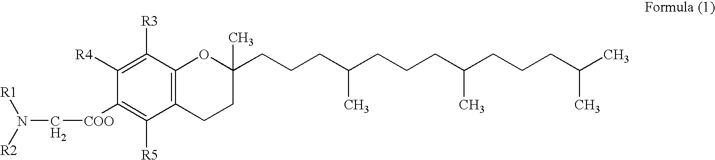
wherein R1 and R2 are the same or different and are each a lower alkyl group or a hydrogen atom with the proviso that they are not hydrogen atoms at the same time, and R3, R4 and R5 are the same or different and are each a hydrogen atom or a methyl group.

(11) The agent for skin external use as stated in (10), wherein the tocopherol glycine ester derivative is N,N-dimethylglycine ester of tocopherol or tocopherol sarcosine ester provided that in the formula (1), R1 and R2, which are simultaneously not hydrogens, are identically or differently a methyl group or hydrogen.

(12) The agent for skin external use as stated in any one of (1) to (11), wherein the component (A) is contained in an amount of 0.1 to 10% by mass.

(13) The agent for skin external use as stated in any one of (1) to (12), wherein the component (B) is a sodium salt of the ascorbic acid-2-phosphoric acid ester.

(14) The agent for skin external use as stated in any one of (1) to (12), wherein the component (B) is a magnesium salt of the ascorbic acid-2-phosphoric acid ester.

(15) The agent for skin external use as stated in any one of (1) to (12), wherein the ascorbic acid-2-phosphoric acid ester derivative is a compound represented by the formula (2): 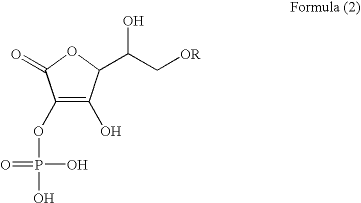
wherein R is an acyl group derived from a higher fatty acid.

(16) The agent for skin external use as stated in any one of (1) to (12), wherein the component (B) is an ascorbic acid-2-phosphoric acid ester-6-palmitic acid ester sodium salt.

(17) The agent for skin external use as stated in any one of (1) to (12), wherein the component (B) is an ascorbic acid-2-phosphoric acid ester-6-hexyldecanoic acid ester sodium salt.

(18) The agent for skin external use as stated in any one of (1) to (17), wherein the component (B) is contained in an amount of 0.1 to 10% by mass.

(19) The agent for skin external use as stated in any one of (1) to (18), wherein the component (C) is a surfactin and/or a salt thereof.

(20) The agent for skin external use as stated in (19), wherein the component (C) is a sodium salt of a surfactin.

(21) The agent for skin external use as stated in (19) or (20), wherein the surfactin contains one or more compounds represented by the formula (3): 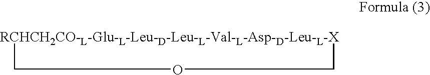
wherein X is an amino acid residue selected from the group consisting of leucine, isoleucine, valine, glycine, serine, alanine, threonine, asparagine, glutamine, aspartic acid, glutamic acid, lysine, arginine, cysteine, methionine, phenylalanine, tyrosine, tryptophan, histidine, proline, 4-hydroxyproline and homoserine, and R is a normal alkyl group of 8 to 14 carbon atoms, an isoalkyl group of 8 to 14 carbon atoms or an anteisoalkyl group of 8 to 14 carbon atoms.

(22) The agent for skin external use as stated in (21), wherein X in the formula (3) is leucine, isoleucine or valine.

(23) The agent for skin external use as stated in any one of (1) to (22), which agent further comprises (D) water.

(24) The agent for skin external use as stated in any one of (1) to (23) which agent is a cosmetic.

### PREFERRED EMBODIMENTS OF THE INVENTION

The present invention is described in detail hereinafter.

(A) Tocopherol Glycine Esters

In the agent for skin external use of the invention, at least one substance selected from the group consisting of tocopherol glycine ester, a tocopherol glycine ester derivative and a salt thereof is contained. That is, the tocopherol glycine ester, a tocopherol glycine ester derivative and a salt thereof may be used singly or in combination with two or more in the agent for skin external use of the invention.

Then, the tocopherol glycine esters (tocopherol glycine ester, tocopherol glycine ester derivatives and salts thereof) are first described.

The tocopherol glycine ester derivative for use in the invention is, for example, a compound which can be represented by the formula (1): 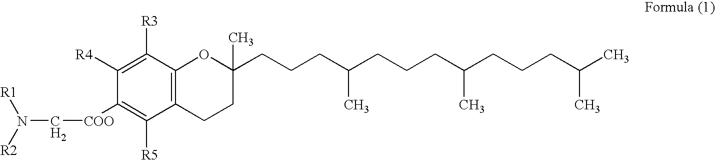
Wherein R1 and R2 are the same or different and are each a lower alkyl group or a hydrogen atom with the proviso that they are not hydrogen atoms at the same time, and R3, R4 and R5 are the same or different and are each a hydrogen atom or a methyl group.

On the other hand, the tocopherol glycine ester for use in the invention is a compound of the above formula (1) wherein R1 and R2 are each a hydrogen atom.

As is apparent from the formula (1), the tocopherol glycine ester and the tocopherol glycine ester derivative have a glycine skeleton or a glycine derivative skeleton, and a tocopherol skeleton.

Examples of tocopherol employable for the tocopherol skeleton include α-tocopherol (in the formula (1), R3, R4 and R5 are each a methyl group), β-tocopherol (in the formula (1), R3 and R5 are each a methyl group, and R4 is a hydrogen atom), γ-tocopherol (in the formula (1), R3 and R4 are each a methyl group, and R5 is a hydrogen atom) and δ-tocopherol (in the formula (1), R3 is a methyl group, and R4 and R5 are each a hydrogen atom). Of these, α-tocopherol, γ-tocopherol or δ-tocopherol is preferably employed.

The above tocopherol has asymmetric carbon at the 2-position of chromanol ring, so that stereoisomers, such as d-form and dl-form, exist. In the present invention, any of these isomers is available, but from the viewpoint of appearance of physiological activity, d-form is particularly preferable.

That is to say, examples of the tocopherol employable for the tocopherol skeleton include dl-α-tocopherol, d-α-tocopherol, d-γ-tocopherol and d-δ-tocopherol. Of these, d-α-tocopherol, d-γ-tocopherol and d-δ-tocopherol are more preferable.

In the glycine derivative skeleton, the lower alkyl group in the definition of R1 or R2 in the formula (1) is, for example, a straight-chain or branched alkyl group of 1 to 6 carbon atoms. Examples of such alkyl groups include methyl, ethyl, n-propyl, n-butyl, isopropyl, isobutyl, 1-methylpropyl, tert-butyl, n-pentyl, 1-ethylpropyl, isoamyl and n-hexyl. Of these, methyl is particularly preferable.

That is to say, preferred examples of glycines to constitute the tocopherol glycine ester derivative for use in the invention include dimethylglycine and sarcosine. On the other hand, the glycine skeleton of the tocopherol glycine ester for use in the invention is constituted of glycine. In the present invention, it is preferable to use a salt of the tocopherol glycine ester or a salt of the tocopherol glycine ester derivative as the tocopherol glycine esters. More specifically, an organic acid salt thereof or an inorganic acid salt thereof is preferable. More preferable is an inorganic salt thereof, and much more preferable is a hydrohalogenic acid salt thereof. Preferred examples of the hydrohalogenic acid salts include a HCl salt and a HBr salt. Of these, a HCl salt is more preferable taking into consideration advantages that the solubility in water is increased and the characteristics are made powdery to thereby facilitate handling. These salts may be used singly or in combination with two or more.

For preparing the tocopherol glycine esters, various processes are thought. A typical example of the process is described below.

Tocopherol represented by the formula (4): 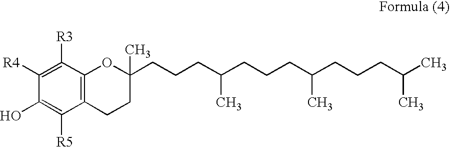
wherein R3, R4 and R5 are the same or different and are each a hydrogen atom or a methyl group,

and glycine or any one of a glycine derivative represented by the formula (5): 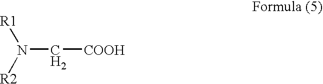
wherein R1 and R2 are the same or different and are each a lower alkyl group or a hydrogen atom with the proviso that they are not hydrogen atoms at the same time,

a reactive acid derivative thereof and a salt thereof such as a hydrohalogenic acid salt are subjected to esterification reaction in a usual way, whereby the tocopherol glycine esters can be readily obtained.

When esterification is directly carried out using glycine or a free glycine derivative, it is preferable to perform the reaction in the presence of an active esterification reagent (dehydrating agent) such as dicyclohexylcarbodiimide or N,N-disuccinimide oxalate. In this case, pyridine is most preferably used as a solvent.

When a glycine derivative represented by the formula (5) and having a substituent on the N atom is used, it is preferable to perform the reaction using an aminoalkylcarboxylic acid wherein an amino group is protected by, for example, a N-tert-butoxycarbonyl (BOC) group, benzyloxycarbonyl group or a 2-nitrobenzenesulfonyl group and to remove the protective group after completion of the reaction, if desired.

In the process using a reactive acid derivative, it is preferable to use an acid halide, particularly an acid chloride.

In the preparation of a hydrohalogenic acid salt of tocopherol glycine ester or a hydrohalogenic acid salt of a tocopherol glycine ester derivative, the hydrohalogenic acid salt may be prepared by a conventional process such as a process comprising temporarily preparing an ester and then allowing it to react with a hydrohalogenic acid (gas phase or solution), or a hydrohalogenic acid salt of glycine or a hydrohalogenic acid salt of a glycine derivative represented by the formula (5) may be used as a starting material.

The tocopherol glycine esters thus obtained are superior to tocopherol alone in the solubility and emulsifiability. Further, when they are applied to agents for skin external use, they are easily hydrolyzed by esterase or carboxyl esterase present in the skin tissues to produce active free tocopherol.

Therefore, it is possible to use the tocopherol glycine esters as active ingredients of agents for skin external use which are expected to exhibit efficacy and effects in the anti-oxidation action, biomembrane stabilizing action, immunity activation action and blood circulation acceleration action, etc.

The tocopherol glycine esters are used so that the content thereof in the agent for skin external use of the invention should be in the range of usually 0.1 to 10% by mass, preferably 0.1 to 5% by mass, more preferably 0.1 to 2% by mass. When the tocopherol glycine esters are contained in the above amount in the agent for skin external use, their efficacy and effects can be well exerted.

(B) Ascorbic Acid-2-Phosphoric Acid Esters

The agent for skin external use of the invention further contains (B) at least one substance selected from the group consisting of ascorbic acid-2-phosphoric acid ester, an ascorbic acid-2-phosphoric acid ester derivative and a salt thereof (also referred to as “ascorbic acid-2-phosphoric acid esters” hereinafter), in addition to the tocopherol glycine esters (A).

The ascorbic acid-2-phosphoric acid ester is a publicly known material and is easily obtainable. The ascorbic acid-2-phosphoric acid ester derivative is, for example, a compound represented by the formula (2): 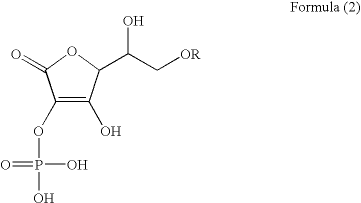
wherein R is an acyl group derived from a higher fatty acid.

The higher fatty acid referred to in the formula (2) means a higher fatty acid of 8 to 21 carbon atoms, and examples thereof include caprylic acid, capric acid, lauric acid, myristic acid, palmitic acid, stearic acid, arachic acid, behenic acid, 2-butylhexanoic acid, 2-hexyldecanoic acid and 2-heptylundecanoic acid. Of these, preferable are palmitic acid and 2-hexyldecanoic acid.

The ascorbic acid-2-phosphoric acid ester and the ascorbic acid-2-phosphoric acid ester derivative may be each D-form, L-form or DL form, but preferable is L-form.

As the salt of the ester or the ester derivative, there can be mentioned a compound wherein a phosphoric acid ester is linked to the 2-position of ascorbic acid and a phosphoric acid residue of the phosphoric acid ester and a base form a salt and/or a compound wherein a higher fatty acid is ester linked to the 6-position and a phosphoric acid residue of the phosphoric acid ester and a base form a salt.

In the present invention, the ascorbic acid-2-phosphoric acid ester, the ascorbic acid-2-phosphoric ester derivative and the salt thereof may be used singly or as a mixture of two or more kinds. Of these, the salt is preferable. The salt is described below.

Examples of salts of the ascorbic acid-2-phosphoric acid ester or salts of the higher fatty acid esters of the ascorbic acid-2-phosphoric acid ester represented by the formula (2) include alkali metal salts, alkaline earth metal salts, Zn salts, Al salts and Ti salts. Of these, preferable are Na salts, K salts, Mg salts and Zn salts, and more preferable are Na salts and Mg salts. More specifically, there can be mentioned, for example, ascorbic acid-2-phosphoric acid ester sodium salt, ascorbic acid-2-phosphoric acid ester magnesium salt, ascorbic acid-2-phosphoric acid ester-6-palmitic acid ester sodium salt, ascorbic acid-2-phosphoric acid ester-6-hexyldecanoic acid ester sodium salt, ascorbic acid-2-phosphoric acid ester-6-palmitic acid ester magnesium salt, and ascorbic acid-2-phosphoric acid ester-6-hexyldecanoic acid ester magnesium salt. These salts may be used singly or in combination of two or more kinds.

The ascorbic acid-2-phosphoric acid esters are used so that the content thereof in the agent for skin external use of the invention should be in the range of usually 0.1 to 10% by mass, preferably 0.5 to 10% by mass, more preferably 1 to 8% by mass. When the ascorbic acid-2-phosphoric acid esters are contained in the above amount, their efficacy and effects can be well exerted.

(C) Surface Active Agent Having Lipopeptide Structure

In the agent for skin external use of the invention, a specific amount of an anionic surface active agent having a lipopeptide structure is further contained in addition to the tocopherol glycine esters (A) and the ascorbic acid-2-phosphoric acid esters (B).

Examples of the anionic surface active agents having a lipopeptide structure include surfactin and/or salts thereof. Preferable are salts of surfactin.

The “surfactin” used herein means a compound represented by the following formula (3) and/or its analogous compound,

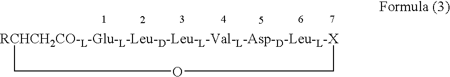

or a composition containing two or more of such compounds.

In the formula (3), X is an amino acid residue selected from the group consisting of leucine, isoleucine, valine, glycine, serine, alanine, threonine, asparagine, glutamine, aspartic acid, glutamic acid, lysine, arginine, cysteine, methionine, phenylalanine, tyrosine, tryptophan, histidine, proline, 4-hydroxyproline and homoserine. In these, X is preferably leucine, isoleucine or valine.

R is a normal alkyl group of 8 to 14 carbon atoms, an isoalkyl group of 8 to 14 carbon atoms or an anteisoalkyl group of 8 to 14 carbon atoms. The normal alkyl group is a straight-chain alkyl group, the isoalkyl group generally has a structure of (CH₃)₂CH—(CH₂)ₙ—, and the anteisoalkyl group generally has a structure of CH₃—CH₂—CH(CH₃)—(CH₂)ₙ—.

The analogous compound means a compound wherein a part of amino acids in the formula (3) are replaced with other amino acids. More specifically, there can be mentioned a compound wherein L-leucine as the second amino acid, L-valine as the fourth amino acid, D-leucine as the sixth amino acid, and the like are replaced with other amino acids, without limiting thereto.

The surfactin is usually produced from prokaryotes. As the prokaryotes, bacillus genus microorganisms, such as bacillus subtilis IAM 1213 strain, IAM 1069 strain, IAM 1259 strain, IAM 1260 strain, IFO 3035 strain and ATCC 21332 strain, are generally employed.

The surfactin can be readily obtained by culturing the microorganisms and purifying them. The purification is carried out by, for example, adding hydrochloric acid to a culture solution to make it acidic, filtering off surfactin precipitated, dissolving them in an organic solvent such as methanol and then subjecting the resulting solution to appropriate various treatments, such as ultrafiltration, activated carbon treatment and crystallization. The precipitation by the addition of acid may be replaced with precipitation by the addition of calcium salt (Biochem. Bioph. Res. Commun., 31, 488-494 (1968)).

As the surfactin, not only surfactin produced from the prokaryotes such as the above-mentioned bacillus genus microorganisms but also surfactin obtained by other processes such as chemical synthesis process is employable.

As can be seen from the formula (3), the surfactin can be used as an inorganic salt or an organic salt. As metals that become counter ions, alkali metals, such as sodium, potassium and lithium, alkaline earth metals, such as calcium and magnesium, and metals that form salts together with the surfactin are employable irrespective of type. Examples of the organic salts include trimethylamine, triethylamine, tributylamine, monoethanolamine, diethanolamine, triethanolamine, lysine, arginine and choline. Of these, sodium salts are particularly preferable.

The sodium salt of surfactin employable herein is, for example, one put in the market by Showa Denko K.K. as sodium surfactin under the trade name of Aminofect (R) (Showa Denko K.K. registered trademark).

The surfactin and/or a salt thereof is used so that the content thereof in the agent for skin external use of the invention should be in the range of usually 0.03 to 20% by mass, preferably 0.1 to 20% by mass, more preferably 0.5 to 12.5% by mass. When the surfactin and/or a salt thereof is contained in the above amount in the agent for skin external use, stability of the tocopherol glycine esters (A) is enhanced, and even in the presence of the ascorbic acid-2-phosphoric acid esters (B), occurrence of coloring (including turbidity) or precipitation with time is inhibited, and efficacy and effects of the agent for skin external use can be maintained over a long period of time.

With respect to the relation to the tocopherol glycine esters (A), the surfactin and/or a salt thereof is desirably contained in an amount of 6 to 1000 parts by mass, preferably 10 to 500 parts by mass, based on 100 parts by mass of the tocopherol glycine esters (A). When it is contained in this amount, an effect of improving stability of the tocopherol glycine esters (A) is conspicuous.

(D) Other Components

To the agent for skin external use of the invention, components which are generally used for agents for skin external use can be added within limits not detrimental to the effects of the present invention.

Examples of such components include:

hydrocarbons such as ozokerite, α-olefin oligomer, light isoparaffin, light liquid isoparaffin, squalene, squalane, synthetic squalane, phytosqualane, ceresin, paraffin, polyethylene powder, polybutene, microcrystalline wax, liquid isoparaffin, liquid paraffin, mineral oil and vaseline;

natural waxes such as jojoba oil, carnauba wax, candelilla wax, rice bran wax, shellac, lanolin, mink sebaceous wax, spermaceti wax, sugarcane wax, sperm whale oil, beeswax and montan wax, natural fats and fatty oils such as avocado oil, almond oil, olive oil, extra virgin olive oil, sesame seed oil, rice bran oil, rice oil, rice germ oil, corn oil, safflower oil, soybean oil, maize oil, rape seed oil, persic oil, palm kernel oil, palm oil, castor oil, sunflower oil, high oleic sunflower oil, grape seed oil, cotton seed oil, coconut oil, hydrogenated coconut oil, beef tallow, hydrogenated oil, horse oil, mink oil, yolk oil, yolk fat oil, rose hip oil, kukui nut oil, evening primrose oil, wheat germ oil, peanut oil, Camellia jeponica oil, Camellia kissi oil, cacao butter, Japan wax, beef bone tallow, nest's-foot oil, swine tallow, equine tallow, ovine tallow, shea butter, macadamia nut oil and meadowfoam seed oil;

fatty acids such as lauric acid, myristic acid, palmitic acid, stearic acid, behenic acid, oleic acid, linoleic acid, linolenic acid, γ-linolenic acid, isostearic acid, 12-hydroxystearic acid, undecylenic acid and coconut oil fatty acid;

higher alcohols such as isostearyl alcohol, octyl dodecanol, hexyl decanol, cholesterol, phytosterol, lauryl alcohol, myristyl alcohol, cetyl alcohol, stearyl alcohol, oleyl alcohol, behenyl alcohol and cetostearyl alcohol;

alkylglyceryl ethers such as batyl alcohol, chimyl alcohol, serachyl alcohol and isostearyl glyceryl ether;

esters such as isopropyl myristate, butyl myristate, isopropyl palmitate, ethyl stearate, butyl stearate, ethyl oleate, ethyl linoleate, isopropyl linoleate, cetyl caprylate, hexyl laurate, isooctyl myristate, decyl myristate, myristyl myristate, cetyl-myristate, octadecyl myristate, cetyl palmitate, stearyl stearate, decyl oleate, oleyl oleate, cetyl ricinoleate, isostearyl laurate, isotridecyl myristate, isocetyl myristate, isostearyl myristate, octyldodecyl myristate, 2-ethylhexyl palmitate, isocetyl palmitate, isostearyl palmitate, 2-ethylhexyl stearate, isocetyl stearate, isodecyl oleate, octyldodecyl oleate, octyldodecyl ricinoleate, ethyl isostearate, isopropyl isostearate, cetyl 2-ethylhexanoate, cetostearyl 2-ethylhexanoate, stearyl 2-ethylhexanoate, hexyl isostearate, ethylene glycol dioctanoate, ethylene glycol dioleate, propylene glycol dicaprylate, propylene glycol dicaprylate/dicaprate, propylene glycol dicaprate, propylene glycol dioleate, neopentyl glycol dicaprate, neopentyl glycol dioctanoate, glyceryl tricaprylate, glyceryl tri 2-ethyl hexanoate, glyceryl tricaprylate/tricaprate, glyceryl tricaprylate/tricaprate/tristearate, glyceryl triundecylate, glyceryl triisopalmitate, glyceryl triisostearate, trimethylolpropane tri 2-ethylhexanoate, trimethylolpropane triisostearate, pentaerythrityl tetra 2-ethylhexanoate, pentaerythrityl tetramyristate, pentaerythrityl tetraisostearate, diglyceryl tetraisostearate, octyldodecyl neopentanoate, isocetyl octanoate, isostearyl octanoate, 2-ethylhexyl isopelargonate, hexyldecyl dimethyloctanoate, octyldodecyl dimethyloctanoate, 2-ethylhexyl isopalmitate, isocetyl isostearate, isostearyl isostearate, octyldodecyl isostearate, lauryl lactate, myristyl lactate, cetyl lactate, octyldodecyl lactate, triethyl citrate, acetyltriethyl citrate, acetyltributyl citrate, trioctyl citrate, triisocetyl citrate, trioctyldodecyl citrate, diisostearyl malate, 2-ethylhexyl hydroxystearate, di 2-ethylhexyl succinate, diisopropyl adipate, diisobutyl adipate, dioctyl adipate, diheptylundecyl adipate, diethyl sebacate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoyl hydroxystearate, stearyl 12-stearoyl hydroxystearate, isostearyl 12-stearoyl hydroxystearate, polyoxyethylene (3) polyoxypropylene (1) cetyl ether acetate, polyoxyethylene (3) polyoxypropylene (1) isocetyl ether acetate, isononyl isononanoate, octyl isononanoate, tridecyl isononanoate and isotridecyl isononanoate;

silicone oils such as methyl polysiloxane, methylphenyl polysiloxane, methyl hydrogen polysiloxane, methyl cyclopolysiloxane, octamethyl cyclotetrasiloxane, decamethyl cyclopentasiloxane, dodecamethyl cyclohexasiloxane, octamethyl trisiloxane, decamethyl tetrasiloxane, tetradecamethyl hexasiloxane, highly polymerized methyl polysiloxane, dimethylsiloxane-methyl(polyoxyethylene)siloxane-methyl(polyoxypropylene)siloxane copolymer, dimethylsiloxane-methyl(polyoxyethylene)siloxane copolymer, dimethylsiloxane-methyl(polyoxypropylene)siloxane copolymer, dimethylsiloxane-methylcetyl oxysiloxane copolymer, dimethylsiloxane-methyl stearoxysiloxane copolymer, polyether modified silicone, alcohol modified silicone, alkyl modified silicone and amino modified silicone;

polyhydric alcohols such as ethylene glycol, diethylene glycol, triethylene glycol, polyethylene glycol, propylene glycol, dipropylene glycol, polypropylene glycol, glycerin, diglycerin, polyglycerin, 3-methyl-1,3-butanediol, 1,3-butanediol, 1,2-pentanediol and 1,2-hexanediol;

saccharides such as mannitol, sorbitol, xylitol, maltitol, erythritol, pentaerythritol, glucose, sucrose, fructose, lactose, maltose, xylose and trehalose;

polymers such as sodium alginate, carrageen, agar, furcellaran, guar gum, quince seed, Amorphophalus konjak (arum root) mannan, tamarind gum, tara gum, dextrin, starch, locust bean gum, gum arabic, gum gatti, karaya gum, gum tragacanth, arabinogalactan, pectin, quince, chitosan, starch, curdlan, xanthan gum, gellan gum, cyclodextrin, dextran, pullulan, microcrystalline cellulose, methyl cellulose, ethyl cellulose, hydroxyethyl cellulose, hydroxypropyl cellulose, hydroxypropylmethyl cellulose, carboxymethyl cellulose, carboxy starch, cationized cellulose, starch phosphate ester, cationized guar gum, carboxymethyl-hydroxypropylated guar gum, hydroxypropylated guar gum, albumin, casein, gelatin, sodium polyacrylate, polyacrylic amide, carboxyvinyl polymer, polyethylene imine, highly polymerized polyethylene glycol, polyvinyl alcohol, polyvinyl pyrrolidone, polyvinyl ether, polyacryl amide, acrylic acid polymer, methacrylic acid polymer, maleic acid polymer, vinylpyridine polymer, ethylene/acrylic acid copolymer, vinyl pyrrolidone based polymer, vinyl alcohol/vinyl pyrrolidone copolymer, nitrogen-substituted acrylamide based polymer, cationized polymer, dimethylacryl ammonium based polymer, acrylic acid based anion polymer, methacrylic acid based anion polymer, acrylate/methacrylate alkyl (C 10 to 30) copolymer and polyoxyethylene/polyoxypropylene copolymer;

alcohols such as ethanol, isopropyl alcohol, 1-butanol, 2-butanol and benzyl alcohol;

anionic surfactants such as coconut oil fatty acid potassium, coconut oil fatty acid sodium, coconut oil fatty acid triethanolamine, potassium laurate, sodium laurate, triethanolamine laurate, potassium myristate, sodium myristate, isopropanolamine myristate, potassium palmitate, sodium palmitate, isopropanolamine palmitate, potassium stearate, sodium stearate, triethanolamine stearate, potassium oleate, sodium oleate, castor oil fatty acid sodium, zinc undecylate, zinc laurate, zinc myristate, magnesium myristate, zinc palmitate, zinc stearate, calcium stearate, magnesium stearate, aluminum stearate, calcium myristate, magnesium myristate, aluminum dimyristate, aluminum isostearate, polyoxyethylene lauryl ether acetate, sodium polyoxyethylene lauryl ether acetate, polyoxyethylene tridecyl ether acetate, sodium polyoxyethylene tridecyl ether acetate, sodium stearoyl lactate, sodium isostearoyl lactate, sodium lauroyl sarcosine, coconut oil fatty acid sarcosine, sodium coconut oil fatty acid sarcosine, coconut oil fatty acid sarcosine triethanolamine, lauroyl sarcosine, potassium lauroyl sarcosine, lauroyl sarcosine triethanolamine, oleoyl sarcosine, sodium myristoyl sarcosine, sodium stearoyl glutamate, coconut oil fatty acid acyl glutamic acid, potassium coconut oil fatty acid acyl glutamate, sodium coconut oil fatty acid acyl glutamate, coconut oil fatty acid acyl glutamate triethanolamine, lauroylacyl glutamic acid, potassium lauroylacyl glutamate, sodium lauroylacyl glutamate, lauroylacyl glutamate triethanolamine, myristoylacyl glutamic acid, potassium myristoylacyl glutamate, sodium myristoylacyl glutamate, stearoylacyl glutamic acid, potassium stearoylacyl glutamate, disodium stearoylacyl glutamate, sodium hydrogenated beef tallow fatty acid acyl glutamate, sodium coconut oil fatty acid/hydrogenated beef tallow fatty acid acyl glutamate, sodium coconut oil fatty acid methylalanine, lauroyl methylalanine, sodium lauroyl methylalanine, lauroyl methylalanine triethanolamine, sodium myristoyl methylalanine, sodium lauroyl methyltaurine, potassium coconut oil fatty acid methyltaurine, sodium coconut oil fatty acid methyltaurine, magnesium coconut oil fatty acid methyltaurine, sodium myristoyl methyltaurine, sodium palmitoyl methyltaurine, sodium stearoyl methyltaurine, sodium oleoyl methyltaurine, sodium alkane sulfonate, sodium tetradecene sulfonate, sodium sulfosuccinate dioctyl, disodium lauryl sulfosuccinate, sodium coconut oil fatty acid ethyl ester sulfonate, sodium lauryl sulfate, triethanolamine lauryl sulfate, sodium cetyl sulfate, triethanolamine alkyl (11,13,15) sulfate, sodium alkyl (12,13) sulfate, triethanolamine alkyl (12,13) sulfate, alkyl (12,14,16) ammonium sulfate, diethanolamine alkyl (12 to 13) sulfate, triethanolamine alkyl (12 to 14) sulfate, triethanolamine alkyl (12 to 15) sulfate, magnesium coconut oil alkyl sulfate/triethanolamine, lauryl ammonium sulfate, potassium lauryl sulfate, magnesium lauryl sulfate, monoethanolamine lauryl sulfate, diethanolamine lauryl sulfate, sodium myristyl sulfate, sodium stearylsulfate, sodium oleyl sulfate, triethanolamine oleyl sulfate, sodium polyoxyethylene lauryl ether sulfate, triethanolamine polyoxyethylene lauryl ether sulfate, sodium polyoxyethylene (1) alkyl (11,13,15) ether sulfate, triethanolamine polyoxyethylene (1) alkyl (11,13,15) ether sulfate, sodium polyoxyethylene (3) alkyl (11 to 15) ether sulfate, sodium polyoxyethylene (2) alkyl (12,13) ether sulfate, sodium polyoxyethylene (3) alkyl (12 to 14) ether sulfate, sodium polyoxyethylene (3) alkyl (12 to 15) ether sulfate, sodium polyoxyethylene (2) lauryl ether sulfate, sodium polyoxyethylene (3) myristyl ether sulfate, sodium higher fatty acid alkanol amide sulfate ester, lauryl phosphate, sodium lauryl phosphate, potassium cetyl phosphate, diethanolamine cetyl phosphate, polyoxyethylene oleyl ether phosphate, polyoxyethylene lauryl ether phosphate, sodium polyoxyethylene lauryl ether phosphate, polyoxyethylene cetyl ether phosphate, sodium polyoxyethylene cetyl ether phosphate, polyoxyethylene stearyl ether phosphate, polyoxyethylene oleyl ether phosphate, sodium polyoxyethylene oleyl ether phosphate, polyoxyethylene alkylphenyl ether phosphate, sodium polyoxyethylene alkylphenyl ether phosphate, triethanolamine polyoxyethylene alkylphenyl ether phosphate, polyoxyethylene octyl ether phosphate, polyoxyethylene (10) alkyl (12,13) ether phosphate, polyoxyethylene alkyl (12 to 15) ether phosphate, polyoxyethylene alkyl (12 to 16) ether phosphate, triethanolamine polyoxyethylene lauryl ether phosphate and diethanolamine polyoxyethylene oleyl ether phosphate;

cationic surfactants such as dioctylamine, dimethylstearylamine, trilaurylamine, diethylaminoethylamide stearate, lauryl trimethylammonium chloride, cetyl trimethylammonium chloride, cetyl trimethylammonium bromide, cetyl trimethylammonium saccharin, stearyl trimethylammonium chloride, alkyl (20 to 22) trimethylammonium chloride, lauryl trimethylammonium bromide, alkyl (16,18) trimethylammonium chloride, stearyl trimethylammonium bromide, stearyl trimethylammonium saccharin, alkyl (28) trimethylammonium chloride, di(polyoxyethylene) oleyl methylammonium (2EO) chloride, dipolyoxyethylene stearyl methylammonium chloride, polyoxyethylene (1) polyoxypropylene (25) diethylmethylammonium chloride, tri(polyoxyethylene) stearyl ammonium (5EO) chloride, distearyl dimethylammonium chloride, dialkyl (12 to 15) dimethylammonium chloride, dialkyl (12 to 18) dimethylammonium chloride, dialkyl (14 to 18) dimethylammonium chloride, dicocoyl dimethylaimonium chloride, dicetyl dimethylammonium chloride, isostearyllauryl dimethylammonium chloride, benzalkonium chloride, myristyl dimethylbenzyl ammonium chloride, lauryl dimethyl(ethylbenzyl) ammonium chloride, stearyl dimethylbenzyl ammonium chloride, lauryl pyridinium chloride, cetyl pyridinium chloride, lauroyl cholamino formylmethylpyridinium chloride, stearoyl cholamino formylmethylpyridinium chloride, alkyl isoquinolinium bromide, methyl benzethonium chloride and benzethonium chloride;

ampholytic surfactants such as 2-alkyl-N-carboxymethyl-N-hydroxyethyl imidazolium betaine, alkyldiamino ethylglycine hydrochloride, sodium lauryldiamino ethylglycine, sodium undecyl hydroxyethyl imidazolium betaine, undecyl-N-carboxymethyl imidazolium betaine, disodium coconut oil fatty acid acyl-N-carboxyethyl-N-hydroxyethyl ethylenediamine, disodium coconut oil fatty acid acyl-N-carboxyethoxyethyl-N-carboxyethyl ethylenediamine, disodium coconut oil fatty acid acyl-N-carboxymethoxyethyl-N-carboxymethyl ethylenediamine, sodium laurylamino propionate, sodium laurylamino dipropionate, triethanolamine laurylamino propionate, sodium palm oil fatty acid acyl-N-carboxyethyl-N-hydroxyethyl ethylenediamine, betaine lauryldimethylamino acetate, betaine coconut oil alkyldimethylamino acetate, betaine stearyl dimethylamino acetate, sodium stearyldimethyl betaine, coconut oil fatty acid amidopropyl betaine, palm oil fatty acid amidopropyl betaine, amidopropyl acetate betaine laurate, amidopropyl betaine ricinoleate, stearyl dihydroxyethyl betaine and lauryl hydroxysulfobetaine;

nonionic surfactants such as polyoxyethylene (10) alkyl (12,13) ether, polyoxyethylene lauryl ether, polyoxyethylene cetyl ether, polyoxyethylene stearyl ether, polyoxyethylene oleyl ether, polyoxyethylene (3,7,12) alkyl (12 to 14) ether, polyoxyethylene tridecyl ether, polyoxyethylene myristyl ether, polyoxyethylene-sec-alkyl (14) ether, polyoxyethylene isocetyl ether, polyoxyethylene cetostearyl ether, polyoxyethylene (2,10,20) isostearyl ether, polyoxyethylene oleylcetyl ether, polyoxyethylene (20) arachyl ether, polyoxyethylene octyldodecyl ether, polyoxyethylene behenyl ether, polyoxyethylene octylphenyl ether, polyoxyethylene nonylphenyl ether, polyoxyethylene dinonylphenyl ether, polyoxyethylene (1) polyoxypropylene (1,2,4,8) cetyl ether, polyoxyethylene (5) polyoxypropylene (1,2,4,8) cetyl ether, polyoxyethylene (10) polyoxypropylene (1,2,4,8) cetyl ether, polyoxyethylene (20) polyoxypropylene (1,2,4,8) cetyl ether, polyoxyethylene polyoxypropylene lauryl ether, polyoxyethylene (3) polyoxypropylene (34) stearyl ether, polyoxyethylene (4) polyoxypropylene (30) stearyl ether, polyoxyethylene (34) polyoxypropylene (23) stearyl ether, polyoxyethylene polyoxypropylene cetyl ether, polyoxyethylene polyoxypropylene decyltetradecyl ether, polyethylene glycol monolaurate, ethylene glycol monostearate, polyethylene glycol monostearate, polyethylene glycol monooleate, ethylene glycol fatty acid ester, self-emulsifying ethylene glycol monostearate, diethylene glycol laurate, polyethylene glycol myristate, polyethylene glycol palmitate, diethylene glycol stearate, self-emulsifying polyethylene glycol (2) monostearate, polyethylene glycol isostearate, ethylene glycol dioctanoate, diethylene glycol dilaurate, polyethylene glycol dilaurate, polyethylene glycol (150) dipalmitate, ethylene glycol distearate, diethylene glycol distearate, polyethylene glycol distearate, ethylene glycol dioleate, polyethylene glycol dioleate, polyethylene glycol diricinoleate, polyoxyethylene (20) sorbitan monolaurate, polyoxyethylene (20) sorbitan monopalmitate, polyoxyethylene (6) sorbitan monostearate, polyoxyethylene (20) sorbitan monostearate, polyoxyethylene (20) sorbitan tristearate, polyoxyethylene (6) sorbitan monooleate, polyoxyethylene (20) sorbitan monooleate, polyoxyethylene (20) sorbitan trioleate, polyoxyethylene (20) coconut oil fatty acid sorbitan, polyoxyethylene (10 to 80) sorbitan monolaurate, polyoxyethylene sorbitan tristearate, polyoxyethylene (20) sorbitan isostearate, polyoxyethylene (150) sorbitan tristearate, polyoxyethylene castor oil, polyoxyethylene hydrogenated castor oil, polyoxyethylene (10) hydrogenated castor oil, polyoxyethylene (20) hydrogenated castor oil, polyoxyethylene (40) hydrogenated castor oil, polyoxyethylene (50) hydrogenated castor oil, polyoxyethylene (60) hydrogenated castor oil, lipophilic glycerin monostearate, lipophilic glycerin monooleate, self-emulsifying glycerin monostearate, coconut oil fatty acid glyceryl, glycerin laurate, glyceryl myristate, glyceryl isostearate, glyceryl ricinoleate, glyceryl monohydroxystearate, glycerin oleate, glyceryl linoleate, glyceryl erucate, glyceryl behenate, wheat germ oil fatty acid glyceride, safflower oil fatty acid glyceryl, hydrogenated soybean fatty acid glyceryl, saturated fatty acid glyceride, cotton seed oil fatty acid glyceryl, monomyristate glyceryl monoisostearate, mono tallowate glyceride, monolanolin fatty acid glyceryl, glyceryl sesquioleate, glyceryl distearate, glyceryl diisostearate, glyceryl diarachidate, sorbitan monolaurate, sorbitan monopalmitate, sorbitan monostearate, sorbitan monoisostearate, sorbitan monooleate, sorbitan sesquistearate, sorbitan sesquioleate, sorbitan tristearate, sorbitan trioleate, coconut oil fatty acid sorbitan, sorbitan isostearate, sorbitan sesquiisostearate, sorbitan distearate, diglyceryl isopalmitate, poly(4 to 10)glyceryl monolaurate, poly(10)glyceryl monomyristate, poly(2 to 10)glyceryl monostearate, poly(2 to 10)glyceryl monoisostearate, poly(2 to 10)glyceryl monooleate, diglyceryl sesquioleate, poly(2 to 10)glyceryl diisostearate, poly(6 to 10)glyceryl distearate, diglyceryl triisostearate, poly(10)glyceryl tristearate, poly(10)glyceryl trioleate, poly(2)glyceryl tetraisostearate, decaglyceryl pentastearate, poly(6 to 10)glyceryl pentaoleate, poly(10)glyceryl heptastearate, decaglyceryl decastearate, poly(10)glyceryl decaoleate, concentrated poly(6)glyceryl ricinoleate, sucrose fatty acid ester, coconut oil fatty acid sucrose ester, alkyl glucoside, coconut oil alkyl dimethylamine oxide, lauryl dimethylamine oxide, dihydroxyethyl lauryl dimethylamine oxide, stearyl dimethylamine oxide, oleyl dimethylamine oxide and polyoxyethylene coconut oil alkyl dimethylamine oxide;

natural surfactants such as saponin, lecithin, soybean phospholipid, hydrogenated soybean phospholipid, soybean lysophospholipid, hydrogenated soybean lysophospholipid, yolk lecithin, hydrogenated yolk lysophosphatidylcholine, phosphatidylcholine, phosphatidylethanolamine, phosphatidylserine, sphingophospholipid, sphingomyelin, ganglioside, bile acid, cholic acid, deoxycholic acid, sodium cholate, sodium deoxycholate, spiculisporic acid, rhamnolipid, trehalose lipid, sophorolipid and mannosyl erythritol lipid;

ultraviolet ray absorbers such as: para-aminobenzoic acid derivatives such as para-aminobenzoic acid, ethyl para-aminobenzoate, glyceryl para-aminobenzoate, amyl para-dimethyl aminobenzoate and 2-ethylhexyl para-dimethyl aminobenzoate; cinnamic acid derivatives such as benzyl cinnamate, mono-2-ethyl hexanoate glyceryl dipara-methoxycinnamate, methyl 2,4-diisopropyl cinnamate, ethyl 2,4-diisopropyl cinnamate, potassium para-methoxycinnamate, sodium para-methoxycinnamate, isopropyl para-methoxycinnamate, 2-ethylhexyl para-methoxycinnamate, 2-ethoxyethyl para-methoxycinnamate and ethyl para-ethoxycinnamate; urocanic acid derivatives such as urocanic acid and ethyl urocanate; benzophenone derivatives such as 2,4-dihydroxybenzophenone, 2,2′,4,4′1-tetrahydroxybenzophenone, sodium 2-hydroxy-4-methoxy-5-sulfobenzophenone, 2-hydroxy-4-methoxybenzophenone-5-sulfonate, 2-hydroxy-4-methoxybenzophenone, 2,2′-dihydroxy-4,4′-dimethoxybenzophenone and sodium 2,2′-dihydroxy-4,4′-dimethoxy-5-sulfobenzophenone; salicylic acid derivatives such as ethylene glycol salicylate, salicylate-2-ethylhexyl, phenyl salicylate, benzyl salicylate, p-tert-butylphenyl salicylate, homomethyl salicylate and salicylate-3,3,5-trimethylcyclohexyl; 2-(2′-hydroxy-5′-methoxyphenyl)benzotriazole and 4-tert-butyl-4′-methoxybenzoyl methane;

powders and color materials such as: kaolin, silicic anhydride, magnesium aluminum silicate, sericite, talc, boron nitride, mica, montmorillonite, hemp cellulose powder, wheat starch, silk powder, maize starch; natural dyes such as nitro dyes, azo dyes, nitroso dyes, triphenylmethane dyes, xanthene dyes, quinoline dyes, anthraquinone dyes, indigo dyes, pyrene dyes, phthalocyanine dyes, flavonoid, quinone, porphyrin, water soluble annatto, sepia powder, caramel, guaiazulene, gardenia blue, gardenia yellow, cochineal, shikonin, sodium copper chlorophyllin, paprika dye, safflower red, safflower yellow, laccaic acid and riboflavin butyrate ester; carbon black, yellow iron oxide, black iron oxide, red iron oxide, iron blue, ultramarine blue, zinc oxide, chromium oxide, titanium oxide, black titanium oxide, zirconium oxide, chromium hydroxide, alumina, magnesium oxide, barium sulfate, aluminum hydroxide, calcium carbonate, lithium cobalt titanate, manganese violet and pearl pigment;

plant extracts such as Angelica keiskei extract, Uncaria gambir extract, avocado extract, sweet hydrangea leaf extract, Gynostemma pentaphyllum makino extract, Althaea officinalis extract, Arnica montana extract, oil soluble Arnica montana extract, almond extract, aloe extract, Japanese styrax benzoin extract, Ginkgo biloba extract, Stinging nettle extract, Orris rhizome root extract, fennel extract, turmeric extract, dog rose fruit extract, Echinacea leaf extract, Scutellaria root extract, Phellodendron bark extract, Japanese captis extract, barley extract, okura extract, Hypericum perforatum extract, oil soluble Hypericum perforatum extract, Lamium album extract, oil soluble Lamium album extract, Ononis spinosa root extract, Nasturtium officinale extract, orange extract, orange flower water, seaweed extract, persimmon tannin, pueraria root extract, Japanese valerian extract, cattail extract, Chamomile (matricaria) extract, oil soluble Chamomile (matricaria) extract, Chamomile (matricaria) distillate, Avena sativa (oat) kernel extract, carrot extract, oil soluble carrot extract, carrot oil, Artemisia capillaris extract, Glycyrrhiza glabra (licorice) extract, powdered Glycyrrhiza glabra (licorice) extract, Glycyrrhiza glabra (licorice) extract flavonoid, cantharides tincture, raspberry extract, kiwi extract, cinchona extract, cucumber extract, apricot kernel extract, quince seed extract, gardenia florida extract, Sasa albomarginata extract, Sophora root extract, walnut shell extract, Citrus paradisi (grapefruit) extract, Clematis vitalba leaf extract, black sugar extract, chlorella extract, mulberry bark extract, Cinnamon bark extract, Gentian extract, Geranium herb extract, black tea extract, Nuphar extract, burdock root extract, oil soluble burdock root extract, wheat germ extract, hydrolyzed wheat powder, rice bran extract, fermented rice bran extract, Symphytum officinale (comfrey) extract, Asiasarum root extract, Crocus sativus (saffron) extract, Saponaria officinalis extract, oil soluble salvia extract, Crataegus cuneata fruit extract, Zanthoxylum fruit extract, Lentinus edodes extract, powdered Lentinus edodes extract, Rehmannia root extract, Lithospermum root extract, oil soluble Lithospermum root extract, Perilla herb extract, linden extract, oil soluble Tilia europaea extract, Filipendula extract, Peony root extract, Coix lacryma-jobi extract, ginger extract, oil soluble ginger extract, ginger tincture, Acorus calamus root extract, Betula pendula (birch) extract, oil soluble Betula alba (birch) extract, Betula pendula (birch) sap, Lonicera japonica extract, Equisetum arvense extract, oil soluble Equisetum arvense extract, scordinin, stevia extract, ivy extract, Crataegus oxyacantaha (whitethorn) extract, sambucus extract, Juniperus communis extract, Achillea milefolium extract, oil soluble Achillea milefolium extract, Mentha piperita (peppermint) extract, Salvia officinalis (sage) extract, oil soluble Salvia officinalis (sage) extract, Salvia officinalis (sage) water, Malva Sylvestris (mallow) extract, Apium graveolens (celery) extract, Cnidium officinale extract, Cnidium officinale water, Swertia herb extract, Glycine max (soybean) extract, Jujube extract, thyme extract, green tea extract, tea leaf dry distilled solution, tea seed extract, clove extract, Citrus unshiu peel extract, Camellia japonica extract, Centella asiatica extract, oil soluble walnut extract, duku extract, Terminalia sericea extract, Capsicum tincture, Japanese angelica root extract, oil soluble Japanese angelica root extract, Japanese angelica root water, Calendula officinalis flower extract, oil soluble Calendula officinalis flower extract, soy milk powder, peach seed extract, Bitter orange peel extract, Houttuynia cordata extract, Solanum lycopersicum (tomato) extract, Potentilla tormentilla Schrk (Rosaceae) extract, fermented soybeans extract, Ginseng extract, oil soluble Ginseng extract, Allium sativum (garlic) extract, wild rose extract, oil soluble wild rose extract, malt extract, malt root extract, ophiopogon tuber extract, parsley extract, rye leaf juice concentrate, peppermint distillate, witch hazel distillate, witch hazel extract, rose extract, parietaria extract, Isodonis japonicus extract, Eriobotrya japonica leaf extract, oil soluble Eriobotrya japonica leaf extract, coltsfoot extract, hoelen extract, Ruscus aculeatus root extract, powdered Ruscus aculeatus root extract, grape extract, grape leaf extract, grape water, Hayflower extract, Luffa cylindrica fruit extract, Luffa cylindrica fruit water, Carthamus tinctorius (safflower) extract, oil soluble Tilia platyphyllos extract, linden distillate, Paeonia suffruticosa (peony) extract, Humulus lupulus (hops) extract, oil soluble Humulus lupulus (hops) extract, pine extract, Silybum marianum (milk thistle) extract, Aesculus hippocastanum (horse chestnut) extract, oil soluble Aesculus hippocastanum (horse chestnut) extract, Sapindus mukurossi extract, Melissa officinalis (balm mint) extract, Melilotus officinalis (melilot) extract, Prunus persica (peach leaf extract, oil soluble Prunus persica (peach) leaf extract, bean sprouts extract, Centaurea cyanus flower extract, Centaurea cyanus flower distillate, Eucalyptus globulus extract, Saxifrage extract, Lilium (lily) extract, Coix seed extract, oil soluble Coix seed extract, Artemisia princeps pampanini extract, Artemisia princeps pampanini water, Lavandula angustifolia (lavender) extract, Lavandula angustifolia (lavender) water, apple extract, Ganoderma lucidum extract, Lactuca sativa (lettuce) extract, lemon extract, Astragalus sinicus extract, Rosa centifolia (rose) flower water, Rosemarinus officinalis (rosemary) extract, oil soluble Rosemarinus officinalis (rosemary) extract, Anthemis nobilis extract and Sanguisorba officinalis extract;

amino acids and peptides such as glycine, alanine, valine, leucine, isoleucine, serine, threonine, phenylalanine, tyrosine, tryptophan, cystine, cysteine, methionine, proline, hydroxyproline, aspartic acid, asparagine, glutamic acid, glutamine, arginine, histidine, lysine, γ-aminobutyric acid, DL-pyrrolidonecarboxylic acid, ε-aminocaproic acid, hydrolyzed elastin, water soluble elastin, hydrolyzed collagen, water soluble collagen, casein, glutathione, wheat peptides and soybean peptide;

vitamins and factors acting like a vitamin such as: vitamin A and analogues thereof such as retinol, retinal, retinoic acid, retinol acetate and retinol palmitate; carotenoids such as α-carotene, β-carotene, γ-carotene, δ-carotene, lycopene, zeaxanthin, cryptoxanthin, echinenon and astaxanthin; vitamin B₁ and analogues thereof such as thiamines; vitamin B₂ and analogues thereof such as riboflavin; vitamin B₆ and analogues thereof such as pyridoxine, pyridoxal and pyridoxamine; vitamin B₁₂ and analogues thereof such as cyanocobalamin; folic acids, nicotinic acid, nicotinamide, pantothenic acids, biotins; vitamin C and analogues thereof such as L-ascorbic acid, sodium L-ascorbate, L-ascorbyl stearate, L-ascorbyl palmitate, L-ascorbyl dipalmitate, L-ascorbyl tetraisopalmitate, L-ascorbate sulfate disodium ester and L-ascorbate-2-glucoside; vitamin D and analogues thereof such as ergocalciferol and cholecalciferol; vitamin E and analogues thereof such as d-α-tocopherol, dl-α-tocopherol, dl-α-tocopherol acetate, dl-α-tocopherol succinate, β-tocopherol, γ-tocopherol and d-δ-tocopherol; ubiquinones, vitamin K and analogues thereof, carnitine, ferulic acid, γ-oryzanol, α-lipoic acid and orotic acid;

antiseptic agents such as benzoic acid, sodium benzoate, undecylenic acid, salicylic acid, sorbic acid, potassium sorbate, dehydroacetic acid, sodium dehydroacetate, isobutyl parahydroxybenzoate, isopropyl parahydroxybenzoate, ethyl parahydroxybenzoate, butyl parahydroxybenzoate, propyl parahydroxybenzoate, benzyl parahydroxybenzoate, methyl parahydroxybenzoate, sodium parahydroxybenzoate methyl, phenoxyethanol, light sensitive dye No. 101, light sensitive dye No. 201 and light sensitive dye No. 401;

antioxidizing agents such as butylhydroxyanisole, butylhydroxytoluene, propyl gallate, erythorbic acid, sodium erythorbate, para-hydroxyanisole and octyl gallate;

chelating agents to bind to a metal ion such as trisodium ethylenediamine hydroxyethyl triacetate, edetic acid, disodium edetate, trisodium edetate, tetrasodium edetate, sodium citrate, gluconic acid, phytic acid, sodium polyphosphate and sodium metaphosphate;

moisturizing agents such as hyaluronic acid, sodium hyaluronate, sodium chondroitin sulfate, sodium lactate, sodium pyrrolidone carboxylate, betaine, lactic acid bacteria fermented solution, yeast extract and ceramide;

anti-inflammatory agents such as glycyrrhizic acid, trisodium glycyrrhizinate, dipotassium glycyrrhizinate, monoammonium glycyrrhizinate, β-glycyrrhetinic acid, glycerin glycyrrhetinate, stearyl glycyrrhetinate, lysozyme chloride, hydrocortisone and allantoin;

pH adjusting agents such as sodium hydroxide, potassium hydroxide and triethanolamine;

salts such as sodium chloride, potassium chloride, magnesium chloride and sodium sulfate;

α-hydroxy acids such as citric acid, glycolic acid, tartaric acid and lactic acid;

whitening agents such as arbutin, α-arbutin and placenta extract;

essential oils such as Archangelica officinalis (angelica) oil, Canangium odoratum (ylang ylang) oil, Canarium luzonicum (elemi) oil, orange oil, Chamomilla recutita (matricaria) oil, Anthemis nobilis oil, Elettaria cardamom (cardamon) oil, Acorus calamus (calamus) oil, Ferula galbaniflua (galbanum) oil, Cinnamomum camphora (camphor) oil, Daucus carota (carrot) seed oil, Salvia sclarea (clary sage) oil, Citrus paradisi (grapefruit) oil, Eugenia caryophyllus (clove) oil, Cinnamon bark oil, Coriandrum sativum (coriander) oil, Cupressus sempervirens (cypress) oil, Santalum album (sandalwood) oil, Juniperus virginiana (cedarwood) oil, Cympogon nardus (citronella) oil, Cinnamomum zeylanicum (Cinnamon) leaf oil, Jasmine officinale (jasmine) absolute oil, Juniperus communis (juniper Berry) oil, Zingiber officinale (ginger) extract, Mentha spicata (spearmint) oil, Salvia officinalis (sage) oil, cedar oil, Pelargonium grabeolens (geranium) oil, Thymus vulgaris (thyme) oil, Melaleuca alternifolia (tea tree) oil, Myristica fragrans (nutmeg) oil, Melaleuca qui.viridiflara (niaouli) oil, Citrus aurantium (neroli) oil, pine oil, Ocimum basilicum (basil) oil, Mentha arvensis oil, Pogostemon patchouli (patchouli) oil, Cymbopogon martini (palmarosa) oil, Foeniculum vulgare (fennel) oil, Citrus bigaradia (petitgrain) oil, Piper nigrum (black pepper) oil, Boswellia carterii (frankincense) oil, Vetiveria zizanoides (vetivert) oil, Mentha piperita (peppermint) oil, Citrus bergamia (bergamot) oil, benzoin oil, Aniba rosaeodora (bois de rose) oil, Origanum majorana (marjoram) oil, mandarin oil, Conumiphora myrrha (myrrh) oil, Melissa officinalis (balm mint) oil, Eucalyptus globulus oil, Citrus junos oil, Citrus aurantifolia (lime) oil, Ravensare aromaticum (ravensare) oil, Lavandula latifolia (lavandin) oil, Lavandula angustifolia (lavender) oil, Tilia vulgaris (linden) oil, lemon oil, lemon grass oil, rose oil, Aniba rosaeodora (rosewood) oil, Rosemarinus officinalis (rosemary) oil and Levisticum officinale (lovage) oil;

terpenes such as limonene, pinene, terpinene, terpinolene, myrcene and longifeelene;

fragrance, water, and the like.

Agent for Skin External Use, Cosmetic

The agent for skin external use of the invention contains the tocopherol glycine esters (A), the ascorbic acid-2-phosphoric acid esters (B) and the surface active agent (C) having a lipopeptide structure, and can further contain the aforesaid other components (D) when needed. The most embodiments of the agent for skin external use are cosmetics. In the present specification, the term “cosmetics” is used in a broad sense including skin milk, skin cream, foundation cream, massage cream, cleansing cream, shaving cream, cleansing foam, skin toner, lotion, pack, shampoo, rinse, hair glowing agent, hair tonic, hair dye, hair treatment agent, tooth paste, gargle, permanent waving agent, ointment, bath agent, body soap, etc., irrespective of category, provided that they are brought into contact with the skin when used. Further, the term “cosmetics” is used in a broad sense, irrespective of age or sex of users.

When the agent for skin external use of the invention is a cosmetic, substances generally employable for cosmetics, which are selected from the aforesaid other components (D), can be employed, and in addition thereto, the existing cosmetic materials other than the components (D) can be further employed. For example, there can be employed all the cosmetic materials described in Keshouhin genryou kizyun (Standards of raw materials of cosmetics), second edition, notes, edited by Society of Japanese Pharmacopoeia, 1984 (YAKUJI NIPPO LIMITED.), Keshouhin genryou kizyun-gai seibun kikaku (Standards of raw materials of cosmetics, nonstandard ingredients), under the editorship of Pharmaceutical Affairs Bureau Evaluation and Registration Division, 1993 (YAKUJI NIPPO LIMITED.), Keshouhin genryou kizyun-gai seibun kikaku tsuiho (Standards of raw materials of cosmetics, nonstandard ingredient Supplement), under the editorship of Pharmaceutical Affairs Bureau Evaluation and Registration Division, 1993 (YAKUJI NIPPO LIMITED.), Keshouhin syubetsu kyoka kizyun (Standards of cosmetic classification permission), under the editorship of Pharmaceutical Affairs Bureau Evaluation and Registration Division, 1993 (YAKUJI NIPPO LIMITED.), Keshouhin syubetsu haigou seibun kikaku (Standards of cosmetic classification ingredients), under the editorship of Pharmaceutical Affairs Bureau Evaluation and Registration Division, 1997 (YAKUJI NIPPO LIMITED.), Keshouhin genryou jiten (Dictionary of raw materials of cosmetics), 1991 (Nikko Chemicals Co., Ltd.), and the like.

These cosmetic materials can be used in such amounts that the total of these materials and the aforesaid other components (D) without missing the effect of the present invention.

The agent for skin external use (including the cosmetic) of the invention can be prepared by using the above components in such amounts that the prescribed contents are obtained and by dissolving, mixing or dispersing them in accordance with a conventional process corresponding to the form of the agent or the cosmetic.

### EFFECT OF THE INVENTION

According to the agent for skin external use of the invention, it is possible to enhance stability of the tocopherol glycine esters in spite that the tocopherol glycine esters and the ascorbic acids are contained simultaneously. Further, occurrence of coloring (including turbidity) and/or precipitation with time can be prevented. And efficacy and effects of the agent for skin eternal use can be well exerted over a long period of time. Therefore, the agent for skin external use according to the present invention can be advantageously applied to general agents for skin external use, particularly cosmetics.

### EXAMPLES

The present invention is further described with reference to the following examples, but it should be construed that the invention is in no way limited to those examples.

Materials Used

Tocopherol glycine esters (A1-A12), ascorbic acid-2-phosphoric acid esters (B1-B4) and sodium surfactin used in the following examples are as follows.

(A) Tocopherol Glycine Esters

A1: dl-α-tocopherol dimethylglycine ester hydrochloride

A2: d-α-tocopherol dimethylglycine ester hydrochloride

A3: d-δ-tocopherol dimethylglycine ester hydrochloride

A4: d-γ-tocopherol dimethylglycine ester hydrochloride

A5: dl-α-tocopherol sarcosine ester hydrochloride

A6: d-α-tocopherol sarcosine ester hydrochloride

A7: d-δ-tocopherol sarcosine ester hydrochloride

A8: d-γ-tocopherol sarcosine ester hydrochloride

A9: dl-α-tocopherol glycine ester hydrochloride

A10: d-α-tocopherol glycine ester hydrochloride

A11: d-δ-tocopherol glycine ester hydrochloride

A12: d-γ-tocopherol glycine ester hydrochloride

(B) Ascorbic Acid-2-Phosphoric Acid Esters

B1: ascorbic acid-2-phosphoric acid ester sodium salt

B2: ascorbic acid-2-phosphoric acid ester magnesium salt

B3: ascorbic acid-2-phosphoric acid ester-6-palmitic acid ester sodium salt

B4: ascorbic acid-2-phosphoric acid ester-6-hexyldecanoic acid ester sodium salt

(C) Surfactin

Aminofect (R) (available from Showa Denko K.K.) was used as sodium surfactin.

Evaluation Methods, Evaluation Criteria

In the following examples, evaluation of coloring (turbidity) and precipitation and evaluation of stability of the tocopherol glycine esters (A) were made in accordance with the following methods and evaluation criteria.

(1). Evaluation of Coloring (Turbidity) and Precipitation

The agent was allowed to stand at 40° C. for one month and then was visually observed. When the agent was transparent, it was evaluated as AA; when the agent had slight turbidity, it was evaluated as BB; when the agent had turbidity but was a homogeneous dispersion, it was evaluated as CC; and when the agent suffered precipitation, it was evaluated as DD.

(2) Evaluation of Stability of Tocopherol Glycine Esters (A)

From the measurement results of high performance liquid chromatography, a residue (%) of the tocopherol glycine esters (A) (component (A)) in the agent was calculated. In detail, the concentration of the component (A) in the agent immediately after the preparation and the concentration of the component (A) in the agent after standing still at 40° C. for 1 month were measured by high performance liquid chromatography under the following measuring conditions, and the residue (%) was calculated from the following formula.
Residue (%)=100×[concentration (g/l) of (A) in the agent after standing still at 40° C. for 1 month/concentration (g/l) of (A) in the agent immediately after preparation]

The measuring conditions of the high performance liquid chromatography are as follows.

Column: Shodex ODSpak F-411

Temperature: 40° C.

Eluent: methanol/acetonitrile=7/3 (containing 0.02M acetic acid and 0.02M sodium acetate)

Flow rate: 0.7 ml/min

Detection: UV detector, 283 nm

When the residue determined as above was not less than 90%, the agent was evaluated as AA; when the residue was not less than 75% and less than 90%, the agent was evaluated as BB; when the residue was not less than 60% and less than 75%, the agent was evaluated as CC; and when the residue was less than 60%, the agent was evaluated as DD.

### Examples (1-1) to (1-4)

The components (i) to (ix) shown in Table 1 were homogeneously stirred and blended in accordance with a blending ratio shown in Table 1 and thereby dissolved to obtain a lotion.

The resulting lotion was placed in a closed container and allowed to stand still at 40° C. for 1 month. Then, occurrence of coloring (turbidity) and precipitation and a residue of the tocopherol glycine esters (A) were examined. The results are set forth in Table 3.

### Examples (1-5) to (1-48)

A lotion was prepared in the same manner as in Examples (1-1) to (1-4), except that the component (A) and the component (B) were changed in accordance with a combination shown in Table 2. The lotion was evaluated in the same manner as described above. The results are set forth in Table 3.

TABLE 1

| Component (% by mass) | | | Ex. (1-1) | Ex. (1-2) | Ex. (1-3) | Ex. (1-4) |
|---|---|---|---|---|---|---|
| (i) | (A) | A1 | 0.20 | 0.20 | 0.20 | 0.20 |
| | Tocopherol | | | | | |
| | Glycine | | | | | |
| | esters | | | | | |
| (ii) | (B) Ascorbic | B1 | 3.00 | — | — | — |
| | acid-2- | B2 | — | 3.00 | — | — |
| | phosphoric | B3 | — | — | 3.00 | — |
| | acid esters | B4 | — | — | — | 3.00 |

| | | | | | |
|---|---|---|---|---|---|
| (iii) | Surfactin Na | 1.00 | 1.00 | 1.00 | 1.00 |
| (iv) | 1,3-Butylene | 8.00 | 8.00 | 8.00 | 8.00 |
| | glycol | | | | |
| (v) | Trehalose | 0.05 | 0.05 | 0.05 | 0.05 |
| (vi) | Citric acid | 0.20 | 0.20 | 0.20 | 0.20 |
| (vii) | Sodium citrate | 0.20 | 0.20 | 0.20 | 0.20 |
| (viii) | Methyl para- | 0.10 | 0.10 | 0.10 | 0.10 |
| | hydroxybenzoate | | | | |
| (ix) | Purified water | 87.25 | 87.25 | 87.25 | 87.25 |

TABLE 2

| | | (B) Ascorbic acid-2-phasphoric acid esters | | | |
|---|---|---|---|---|---|
| | | B1 | B2 | B3 | B4 |
| (A) | A1 | Ex. (1-1) | Ex. (1-2) | Ex. (1-3) | Ex. (1-4) |
| Tocopherol | A2 | Ex. (1-5) | Ex. (1-6) | Ex. (1-7) | Ex. (1-8) |
| glycine | A3 | Ex. (1-9) | Ex. (1-10) | Ex. (1-11) | Ex. (1-12) |
| esters | A4 | Ex. (1-13) | Ex. (1-14) | Ex. (1-15) | Ex. (1-16) |
| | A5 | Ex. (1-17) | Ex. (1-18) | Ex. (1-19) | Ex. (1-20) |
| | A6 | Ex. (1-21) | Ex. (1-22) | Ex. (1-23) | Ex. (1-24) |
| | A7 | Ex. (1-25) | Ex. (1-26) | Ex. (1-27) | Ex. (1-28) |
| | A8 | Ex. (1-29) | Ex. (1-30) | Ex. (1-31) | Ex. (1-32) |
| | A9 | Ex. (1-33) | Ex. (1-34) | Ex. (1-35) | Ex. (1-36) |
| | A10 | Ex. (1-37) | Ex. (1-38) | Ex. (1-39) | Ex. (1-40) |
| | A11 | Ex. (1-41) | Ex. (1-42) | Ex. (1-43) | Ex. (1-44) |
| | A12 | Ex. (1-45) | Ex. (1-46) | Ex. (1-47) | Ex. (1-48) |

TABLE 3

| | Ex. (1-1) | Ex. (1-2) | Ex. (1-3) | Ex. (1-4) |
|---|---|---|---|---|
| Coloring (turbidity) | AA | AA | AA | AA |
| precipitation | | | | |
| Stability | AA | AA | AA | AA |
| | Ex. (1-5) | Ex. (1-6) | Ex. (1-7) | Ex. (1-8) |
| Coloring (turbidity) | AA | AA | AA | AA |
| precipitation | | | | |
| Stability | AA | AA | AA | AA |
| | Ex. (1-9) | Ex. (1-10) | Ex. (1-11) | Ex. (1-12) |
| Coloring (turbidity) | AA | AA | AA | AA |
| precipitation | | | | |
| Stability | BB | BB | BB | BB |
| | Ex. (1-13) | Ex. (1-14) | Ex. (1-15) | Ex. (1-16) |
| Coloring (turbidity) | AA | AA | AA | AA |
| precipitation | | | | |
| Stability | BB | BB | BB | BB |
| | Ex. (1-17) | Ex. (1-18) | Ex. (1-19) | Ex. (1-20) |
| Coloring (turbidity) | AA | AA | AA | AA |
| precipitation | | | | |
| Stability | AA | AA | AA | AA |
| | Ex. (1-21) | Ex. (1-22) | Ex. (1-23) | Ex. (1-24) |
| Coloring (turbidity) | AA | AA | AA | AA |
| precipitation | | | | |
| Stability | AA | AA | AA | AA |
| | Ex. (1-25) | Ex. (1-26) | Ex. (1-27) | Ex. (1-28) |
| Coloring (turbidity) | AA | AA | AA | AA |
| precipitation | | | | |
| Stability | BB | BB | BB | BB |
| | Ex. (1-29) | Ex. (1-30) | Ex. (1-31) | Ex. (1-32) |
| Coloring (turbidity) | AA | AA | AA | AA |
| precipitation | | | | |
| Stability | BB | BB | BB | BB |
| | Ex. (1-33) | Ex. (1-34) | Ex. (1-35) | Ex. (1-36) |
| Coloring (turbidity) | AA | AA | AA | AA |
| precipitation | | | | |
| Stability | AA | AA | AA | AA |
| | Ex. (1-37) | Ex. 1-38) | Ex. (1-39) | Ex. (1-40) |
| Coloring (turbidity) | AA | AA | AA | AA |
| precipitation | | | | |
| Stability | AA | AA | AA | AA |
| | Ex. (1-41) | Ex. (1-42) | Ex. (1-43) | Ex. (1-44) |
| Coloring (turbidity) | AA | AA | AA | AA |
| precipitation | | | | |
| Stability | BB | BB | BB | BB |
| | Ex. (1-45) | Ex. (1-46) | Ex. (1-47) | Ex. (1-48) |
| Coloring (turbidity) | AA | AA | AA | AA |
| precipitation | | | | |
| Stability | BB | BB | BB | BB |

### Examples (2-1) to (2-4)

The components (i) to (ix) shown in Table 4 were homogeneously stirred and blended in accordance with a blending ratio shown in Table 4 and thereby dissolved to obtain a lotion.

The resulting lotion was placed in a closed container and allowed to stand still at 40° C. for 1 month. Then, occurrence of coloring (turbidity) and precipitation and a residue of the tocopherol glycine esters (A) were examined. The results are set forth in Table 6.

### Examples (2-5) to (2-48)

A lotion was prepared in the same manner as in Examples (2-1) to (2-4), except that the component (A) and the component (B) were changed in accordance with a combination shown in Table 5. The lotion was evaluated in the same manner as described above. The results are set forth in Table 6.

TABLE 4

| Component (% by mass) | | | Ex. (2-1) | Ex. (2-2) | Ex. (2-3) | Ex. (2-4) |
|---|---|---|---|---|---|---|
| (i) | (A) | A1 | 1.00 | 1.00 | 1.00 | 1.00 |
| | Tocopherol | | | | | |
| | glycine | | | | | |
| | esters | | | | | |
| (ii) | (B) Ascorbic | B1 | 9.00 | — | — | — |
| | acid-2- | B2 | — | 9.00 | — | — |
| | phosphoric | B3 | — | — | 9.00 | — |
| | acid esters. | B4 | — | — | — | 9.00 |

| | | | | | |
|---|---|---|---|---|---|
| (iii) | Surfactin Na | 1.00 | 1.00 | 1.00 | 1.00 |
| (iv) | 1,3-Butylene | 8.00 | 8.00 | 8.00 | 8.00 |
| | glycol | | | | |
| (v) | Trehalose | 0.05 | 0.05 | 0.05 | 0.05 |
| (vi) | Citric acid | 0.20 | 0.20 | 0.20 | 0.20 |
| (vii) | Sodium citrate | 0.20 | 0.20 | 0.20 | 0.20 |
| (viii) | Methyl para- | 0.10 | 0.10 | 0.10 | 0.10 |
| | hydroxybenzoate | | | | |
| (ix) | Purified water | 80.45 | 80.45 | 80.45 | 80.45 |

TABLE 5

| | | (B) Ascorbic acid-2-phosphoric acid esters | | | |
|---|---|---|---|---|---|
| | | B1 | B2 | B3 | B4 |
| (A) | A1 | Ex. (2-1) | Ex. (2-2) | Ex. (2-3) | Ex. (2-4) |
| Tocopherol | A2 | Ex. (2-5) | Ex. (2-6) | Ex. (2-7) | Ex. (2-8) |
| glycine | A3 | Ex. (2-9) | Ex. (2-10) | Ex. (2-11) | Ex. (2-12) |
| esters | A4 | Ex. (2-13) | Ex. (2-14) | Ex. (2-15) | Ex. (2-16) |
| | A5 | Ex. (2-17) | Ex. (2-18) | Ex. (2-19) | Ex. (2-20) |
| | A6 | Ex. (2-21) | Ex. (2-22) | Ex. (2-23) | Ex. (2-24) |
| | A7 | Ex. (2-25) | Ex. (2-26) | Ex. (2-27) | Ex. (2-28) |
| | A8 | Ex. (2-29) | Ex. (2-30) | Ex. (2-31) | Ex. (2-32) |
| | A9 | Ex. (2-33) | Ex. (2-34) | Ex. (2-35) | Ex. (2-36) |
| | A10 | Ex. (2-37) | Ex. (2-38) | Ex. (2-39) | Ex. (2-40) |
| | A11 | Ex. (2-41) | Ex. (2-42) | Ex. (2-43) | Ex. (2-44) |
| | A12 | Ex. (2-45) | Ex. (2-46) | Ex. (2-47) | Ex. (2-48) |

TABLE 6

| | Ex. (2-1) | Ex. (2-2) | Ex. (2-3) | Ex. (2-4) |
|---|---|---|---|---|
| Coloring (turbidity) | BB | BB | BB | BB |
| precipitation | | | | |
| Stability | BB | BB | BB | BB |
| | Ex. (2-5) | Ex. (2-6) | Ex. (2-7) | Ex. (2-8) |
| Coloring (turbidity) | BB | BB | BB | BB |
| precipitation | | | | |
| Stability | BB | BB | BB | BB |
| | Ex. (2-9) | Ex. (2-10) | Ex. (2-11) | Ex. (2-12) |
| Coloring (turbidity) | BB | BB | BB | BB |
| precipitation | | | | |
| Stability | CC | CC | CC | CC |
| | Ex. (2-13) | Ex. (2-14) | Ex. (2-15) | Ex. (2-16) |
| Coloring (turbidity) | BB | BB | BB | BB |
| precipitation | | | | |
| Stability | CC | CC | CC | CC |
| | Ex. (2-17) | Ex. (2-18) | Ex. (2-19) | Ex. (2-20) |
| Coloring (turbidity) | BB | BB | BB | BB |
| precipitation | | | | |
| Stability | BB | BB | BB | BB |
| | Ex. (2-21) | Ex. (2-22) | Ex. (2-23) | Ex. (2-24) |
| Coloring (turbidity) | BB | BB | BB | BB |
| precipitation | | | | |
| Stability | BB | BB | BB | BB |
| | Ex. (2-25) | Ex. (2-26) | Ex. (2-27) | Ex. (2-28) |
| Coloring (turbidity) | BB | BB | BB | BB |
| precipitation | | | | |
| Stability | CC | CC | CC | CC |
| | Ex. (2-29) | Ex. (2-30) | Ex. (2-31) | Ex. (2-32) |
| Coloring (turbidity) | BB | BB | BB | BB |
| precipitation | | | | |
| Stability | CC | CC | CC | CC |
| | Ex. (2-33) | Ex. (2-34) | Ex. (2-35) | Ex. (2-36) |
| Coloring (turbidity) | BB | BB | BB | BB |
| precipitation | | | | |
| Stability | BB | BB | BB | BB |
| | Ex. (2-37) | Ex. (2-38) | Ex. (2-39) | Ex. (2-40) |
| Coloring (turbidity) | BB | BB | BB | BB |
| precipitation | | | | |
| Stability | BB | BB | BB | BB |
| | Ex. (2-41) | Ex. (2-42) | Ex. (2-43) | Ex. (2-44) |
| Coloring (turbidity) | BB | BB | BB | BB |
| precipitation | | | | |
| Stability | CC | CC | CC | CC |
| | Ex. (2-45) | Ex. (2-46) | Ex. (2-47) | Ex. (2-48) |
| Coloring (turbidity) | BB | BB | BB | BB |
| precipitation | | | | |
| Stability | CC | CC | CC | CC |

### Examples (3-1) to (3-4)

The components (i) to (ix) shown in Table 7 were homogeneously stirred and blended in accordance with a blending ratio shown in Table 7 and thereby dissolved to obtain a lotion.

The resulting lotion was placed in a closed container and allowed to stand still at 40° C. for 1 month. Then, occurrence of coloring (turbidity) and precipitation and a residue of the tocopherol glycine esters (A) were examined. The results are set forth in Table 9.

### Examples (3-5) to (3-48)

A lotion was prepared in the same manner as in Examples (3-1) to (3-4), except that the component (A) and the component (B) were changed in accordance with a combination shown in Table 8. The lotion was evaluated in the same manner as described above. The results are set forth in Table 9.

TABLE 7

| Component (% by mass) | | | Ex. (3-1) | Ex. (3-2) | Ex. (3-3) | Ex. (3-4) |
|---|---|---|---|---|---|---|
| (i) | (A) | A1 | 1.00 | 1.00 | 1.00 | 1.00 |
| | Tocopherol | | | | | |
| | glycine | | | | | |
| | esters | | | | | |
| (ii) | (B) Ascorbic | B1 | 1.00 | — | — | — |
| | acid-2- | B2 | — | 1.00 | — | — |
| | phosphoric | B3 | — | — | 1.00 | — |
| | acid esters | B4 | — | — | — | 1.00 |

| | | | | | |
|---|---|---|---|---|---|
| (iii) | Surfactin Na | 1.00 | 1.00 | 1.00 | 1.00 |
| (iv) | 1,3-Butylene | 8.00 | 8.00 | 8.00 | 8.00 |
| | glycol | | | | |
| (v) | Trehalose | 0.05 | 0.05 | 0.05 | 0.05 |
| (vi) | Citric acid | 0.20 | 0.20 | 0.20 | 0.20 |
| (vii) | Sodium citrate | 0.20 | 0.20 | 0.20 | 0.20 |
| (viii) | Methyl para- | 0.10 | 0.10 | 0.10 | 0.10 |
| | hydroxybenzoate | | | | |
| (ix) | Purified water | 88.45 | 88.45 | 88.45 | 88.45 |

TABLE 8

| | | (B) Ascorbic acid-2-phosphoric acid esters | | | |
|---|---|---|---|---|---|
| | | B1 | B2 | B3 | B4 |
| (A) | A1 | Ex. (3-1) | Ex. (3-2) | Ex. (3-3) | Ex. (3-4) |
| Tocopherol | A2 | Ex. (3-5) | Ex. (3-6) | Ex. (3-7) | Ex. (3-8) |
| glycine | A3 | Ex. (3-9) | Ex. (3-10) | Ex. (3-11) | Ex. (3-12) |
| esters | A4 | Ex. (3-13) | Ex. (3-14) | Ex. (3-15) | Ex. (3-16) |
| | A5 | Ex. (3-17) | Ex. (3-18) | Ex. (3-19) | Ex. (3-20) |
| | A6 | Ex. (3-21) | Ex. (3-22) | Ex. (3-23) | Ex. (3-24) |
| | A7 | Ex. (3-25) | Ex. (3-26) | Ex. (3-27) | Ex. (3-28) |
| | A8 | Ex. (3-29) | Ex. (3-30) | Ex. (3-31) | Ex. (3-32) |
| | A9 | Ex. (3-33) | Ex. (3-34) | Ex. (3-35) | Ex. (3-36) |
| | A10 | Ex. (3-37) | Ex. (3-38) | Ex. (3-39) | Ex. (3-40) |
| | A11 | Ex. (3-41) | Ex. (3-42) | Ex. (3-43) | Ex. (3-44) |
| | A12 | Ex. (3-45) | Ex. (3-46) | Ex. (3-47) | Ex. (3-48) |

TABLE 9

| | Ex. (3-1) | Ex. (3-2) | Ex. (3-3) | Ex. (3-4) |
|---|---|---|---|---|
| Coloring (turbidity) | AA | AA | AA | AA |
| precipitation | | | | |
| Stability | AA | AA | AA | AA |
| | Ex. (3-5) | Ex. (3-6) | Ex. (3-7) | Ex. (3-8) |
| Coloring (turbidity) | AA | AA | AA | AA |
| precipitation | | | | |
| Stability | AA | AA | AA | AA |
| | Ex. (3-9) | Ex. (3-10) | Ex. (3-11) | Ex. (3-12) |
| Coloring (turbidity) | AA | AA | AA | AA |
| precipitation | | | | |
| Stability | BB | BB | BB | BB |
| | Ex. (3-13) | Ex. (3-14) | Ex. (3-15) | Ex. (3-16) |
| Coloring (turbidity) | AA | AA | AA | AA |
| precipitation | | | | |
| Stability | BB | BB | BB | BB |
| | Ex. (3-17) | Ex. (3-18) | Ex. (3-19) | Ex. (3-20) |
| Coloring (turbidity) | AA | AA | AA | AA |
| precipitation | | | | |
| Stability | AA | AA | AA | AA |
| | Ex. (3-21) | Ex. (3-22) | Ex. (3-23) | Ex. (3-24) |
| Coloring (turbidity) | AA | AA | AA | AA |
| precipitation | | | | |
| Stability | AA | AA | AA | AA |
| | Ex. (3-25) | Ex. (3-26) | Ex. (3-27) | Ex. (3-28) |
| Coloring (turbidity) | AA | AA | AA | AA |
| precipitation | | | | |
| Stability | BB | BB | BB | BB |
| | Ex. (3-29) | Ex. (3-30) | Ex. (3-31) | Ex. (3-32) |
| Coloring (turbidity) | AA | AA | AA | AA |
| precipitation | | | | |
| Stability | BB | BB | BB | BB |
| | Ex. (3-33) | Ex. (3-34) | Ex. (3-35) | Ex. (3-36) |
| Coloring (turbidity) | AA | AA | AA | AA |
| precipitation | | | | |
| Stability | AA | AA | AA | AA |
| | Ex. (3-37) | Ex. (3-38) | Ex. (3-39) | Ex. (3-40) |
| Coloring (turbidity) | AA | AA | AA | AA |
| precipitation | | | | |
| Stability | AA | AA | AA | AA |
| | Ex. (3-41) | Ex. (3-42) | Ex. (3-43) | Ex. (3-44) |
| Coloring (turbidity) | AA | AA | AA | AA |
| precipitation | | | | |
| Stability | BB | BB | BB | BB |
| | Ex. (3-45) | Ex. (3-46) | Ex. (3-47) | Ex. (3-48) |
| Coloring (turbidity) | AA | AA | AA | AA |
| precipitation | | | | |
| Stability | BB | BB | BB | BB |

### Examples (4-1) to (4-4)

The components (i) to (ix) shown in Table 10 were homogeneously stirred and blended in accordance with a blending ratio shown in Table 10 and thereby dissolved to obtain a lotion.

The resulting lotion was placed in a closed container and allowed to stand still at 40° C. for 1 month. Then, occurrence of coloring (turbidity) and precipitation and a residue of the tocopherol glycine esters (A) were examined. The results are set forth in Table 12.

### Examples (4-5) to (4-48)

A lotion was prepared in the same manner as in Examples (4-1) to (4-4), except that the component (A) and the component (B) were changed in accordance with a combination shown in Table 11. The lotion was evaluated in the same manner as described above. The results are set forth in Table 12.

TABLE 10

| Component (% by mass) | | | Ex. (4-1) | Ex. (4-2) | Ex. (4-3) | Ex. (4-4) |
|---|---|---|---|---|---|---|
| (i) | (A) | A1 | 0.20 | 0.20 | 0.20 | 0.20 |
| | Tocopherol | | | | | |
| | glycine | | | | | |
| | esters | | | | | |
| (ii) | (B) Ascorbic | B1 | 3.00 | — | — | — |
| | acid-2- | B2 | — | 3.00 | — | — |
| | phosphoric | B3 | — | — | 3.00 | — |
| | acid esters | B4 | — | — | — | 3.00 |

| | | | | | |
|---|---|---|---|---|---|
| (iii) | Surfactin Na | 0.10 | 0.10 | 0.10 | 0.10 |
| (iv) | 1,3-Butylene | 8.00 | 8.00 | 8.00 | 8.00 |
| | glycol | | | | |
| (v) | Trehalose | 0.05 | 0.05 | 0.05 | 0.05 |
| (vi) | Citric acid | 0.20 | 0.20 | 0.20 | 0.20 |
| (vii) | Sodium citrate | 0.20 | 0.20 | 0.20 | 0.20 |
| (viii) | Methyl para- | 0.10 | 0.10 | 0.10 | 0.10 |
| | hydroxybenzoate | | | | |
| (ix) | Purified water | 88.15 | 88.15 | 88.15 | 88.15 |

TABLE 11

| | | (B) Ascorbic acid-2-phosphoric acid esters | | | |
|---|---|---|---|---|---|
| | | B1 | B2 | B3 | B4 |
| (A) | A1 | Ex. (4-1) | Ex. (4-2) | Ex. (4-3) | Ex. (4-4) |
| Tocopherol | A2 | Ex. (4-5) | Ex. (4-6) | Ex. (4-7) | Ex. (4-8) |
| glycine | A3 | Ex. (4-9) | Ex. (4-10) | Ex. (4-11) | Ex. (4-12) |
| esters | A4 | Ex. (4-13) | Ex. (4-14) | Ex. (4-15) | Ex. (4-16) |
| | A5 | Ex. (4-17) | Ex. (4-18) | Ex. (4-19) | Ex. (4-20) |
| | A6 | Ex. (4-21) | Ex. (4-22) | Ex. (4-23) | Ex. (4-24) |
| | A7 | Ex. (4-25) | Ex. (4-26) | Ex. (4-27) | Ex. (4-28) |
| | A8 | Ex. (4-29) | Ex. (4-30) | Ex. (4-31) | Ex. (4-32) |
| | A9 | Ex. (4-33) | Ex. (4-34) | Ex. (4-35) | Ex. (4-36) |
| | A10 | Ex. (4-37) | Ex. (4-38) | Ex. (4-39) | Ex. (4-40) |
| | A11 | Ex. (4-41) | Ex. (4-42) | Ex. (4-43) | Ex. (4-44) |
| | A12 | Ex. (4-45) | Ex. (4-46) | Ex. (4-47) | Ex. (4-48) |

TABLE 12

| | Ex. (4-1) | Ex. (4-2) | Ex. (4-3) | Ex. (4-4) |
|---|---|---|---|---|
| Coloring (turbidity) | BB | BB | BB | BB |
| precipitation | | | | |
| Stability | BB | BB | BB | BB |
| | Ex. (4-5) | Ex. (4-6) | Ex. (4-7) | Ex. (4-8) |
| Coloring (turbidity) | BB | BB | BB | BB |
| precipitation | | | | |
| Stability | BB | BB | BB | BB |
| | Ex. (4-9) | Ex. (4-10) | Ex. (4-11) | Ex. (4-12) |
| Coloring (turbidity) | BB | BB | BB | BB |
| precipitation | | | | |
| Stability | CC | CC | CC | CC |
| | Ex. (4-13) | Ex. (4-14) | Ex. (4-15) | Ex. (4-16) |
| Coloring (turbidity) | BB | BB | BB | BB |
| precipitation | | | | |
| Stability | CC | CC | CC | CC |
| | Ex. (4-17) | Ex. (4-18) | Ex. (4-19) | Ex. (4-20) |
| Coloring (turbidity) | BB | BB | BB | BB |
| precipitation | | | | |
| Stability | BB | BB | BB | BB |
| | Ex. (4-21) | Ex. (4-22) | Ex. (4-23) | Ex. (4-24) |
| Coloring (turbidity) | BB | BB | BB | BB |
| precipitation | | | | |
| Stability | BB | BB | BB | BB |
| | Ex. (4-25) | Ex. (4-26) | Ex. (4-27) | Ex. (4-28) |
| Coloring (turbidity) | BB | BB | BB | BB |
| precipitation | | | | |
| Stability | CC | CC | CC | CC |
| | Ex. (4-29) | Ex. (4-30) | Ex. (4-31) | Ex. (4-32) |
| Coloring (turbidity) | BB | BB | BB | BB |
| precipitation | | | | |
| Stability | CC | CC | CC | CC |
| | Ex. (4-33) | Ex. (4-34) | Ex. (4-35) | Ex. (4-36) |
| Coloring (turbidity) | BB | BB | BB | BB |
| precipitation | | | | |
| Stability | BB | BB | BB | BB |
| | Ex. (4-37) | Ex. (4-38) | Ex. (4-39) | Ex. (4-40) |
| Coloring (turbidity) | BB | BB | BB | BB |
| precipitation | | | | |
| Stability | BB | BB | BB | BB |
| | Ex. (4-41) | Ex. (4-42) | Ex. (4-43) | Ex. (4-44) |
| Coloring (turbidity) | BB | BB | BB | BB |
| precipitation | | | | |
| Stability | CC | CC | CC | CC |
| | Ex. (4-45) | Ex. (4-46) | Ex. (4-47) | Ex. (4-48) |
| Coloring (turbidity) | BB | BB | BB | BB |
| precipitation | | | | |
| Stability | CC | CC | CC | CC |

### Examples (5-1) to (5-4)

The components (i) to (ix) shown in Table 13 were homogeneously stirred and blended in accordance with a blending ratio shown in Table 13 and thereby dissolved to obtain a lotion.

The resulting lotion was placed in a closed container and allowed to stand still at 40° C. for 1 month. Then, occurrence of coloring (turbidity) and precipitation and a residue of the tocopherol glycine esters (A) were examined. The results are set forth in Table 15.

### Examples (5-5) to (5-48)

A lotion was prepared in the same manner as in Examples (5-1) to (5-4), except that the component (A) and the component (B) were changed in accordance with a combination shown in Table 14. The lotion was evaluated in the same manner as described above. The results are set forth in Table 15.

TABLE 13

| Component (% by mass) | | | Ex. (5-1) | Ex. (5-2) | Ex. (5-3) | Ex. (5-4) |
|---|---|---|---|---|---|---|
| (i) | (A) | A1 | 1.00 | 1.00 | 1.00 | 1.00 |
| | Tocopherol | | | | | |
| | glycine | | | | | |
| | esters | | | | | |
| (ii) | (B) Ascorbic | B1 | 1.00 | — | — | — |
| | acid-2- | B2 | — | 1.00 | — | — |
| | phosphoric | B3 | — | — | 1.00 | — |
| | acid esters | B4 | — | — | — | 1.00 |

| | | | | | |
|---|---|---|---|---|---|
| (iii) | Surfactin Na | 0.10 | 0.10 | 0.10 | 0.10 |
| (iv) | 1,3-Butylene | 8.00 | 8.00 | 8.00 | 8.00 |
| | glycol | | | | |
| (v) | Trehalose | 0.05 | 0.05 | 0.05 | 0.05 |
| (vi) | Citric acid | 0.20 | 0.20 | 0.20 | 0.20 |
| (vii) | Sodium citrate | 0.20 | 0.20 | 0.20 | 0.20 |
| (viii) | Methyl para- | 0.10 | 0.10 | 0.10 | 0.10 |
| | hydroxybenzoate | | | | |
| (ix) | Purified water | 89.35 | 89.35 | 89.35 | 89.35 |

TABLE 14

| | | (B) Ascorbic acid-2-phosphoric acid esters | | | |
|---|---|---|---|---|---|
| | | B1 | B2 | B3 | B4 |
| (A) | A1 | Ex. (5-1) | Ex. (5-2) | Ex. (5-3) | Ex. (5-4) |
| Tocopherol | A2 | Ex. (5-5) | Ex. (5-6) | Ex. (5-7) | Ex. (5-8) |
| glycine | A3 | Ex. (5-9) | Ex. (5-10) | Ex. (5-11) | Ex. (5-12) |
| esters | A4 | Ex. (5-13) | Ex. (5-14) | Ex. (5-15) | Ex. (5-16) |
| | A5 | Ex. (5-17) | Ex. (5-18) | Ex. (5-19) | Ex. (5-20) |
| | A6 | Ex. (5-21) | Ex. (5-22) | Ex. (5-23) | Ex. (5-24) |
| | A7 | Ex. (5-25) | Ex. (5-26) | Ex. (5-27) | Ex. (5-28) |
| | A8 | Ex. (5-29) | Ex. (5-30) | Ex. (5-31) | Ex. (5-32) |
| | A9 | Ex. (5-33) | Ex. (5-34) | Ex. (5-35) | Ex. (5-36) |
| | A10 | Ex. (5-37) | Ex. (5-38) | Ex. (5-39) | Ex. (5-40) |
| | A11 | Ex. (5-41) | Ex. (5-42) | Ex. (5-43) | Ex. (5-44) |
| | A12 | Ex. (5-45) | Ex. (5-46) | Ex. (5-47) | Ex. (5-48) |

TABLE 15

| | Ex. (5-1) | Ex. (5-2) | Ex. (5-3) | Ex. (5-4) |
|---|---|---|---|---|
| Coloring (turbidity) | BB | BB | BB | BB |
| precipitation | | | | |
| Stability | BB | BB | BB | BB |
| | Ex. (5-5) | Ex. (5-6) | Ex. (5-7) | Ex. (5-8) |
| Coloring (turbidity) | BB | BB | BB | BB |
| precipitation | | | | |
| Stability | BB | BB | BB | BB |
| | Ex. (5-9) | Ex. (5-10) | Ex. (5-11) | Ex. (5-12) |
| Coloring (turbidity) | BB | BB | BB | BB |
| precipitation | | | | |
| Stability | CC | CC | CC | CC |
| | Ex. (5-13) | Ex. (5-14) | Ex. (5-15) | Ex. (5-16) |
| Coloring (turbidity) | BB | BB | BB | BB |
| precipitation | | | | |
| Stability | CC | CC | CC | CC |
| | Ex. (5-17) | Ex. (5-18) | Ex. (5-19) | Ex. (5-20) |
| Coloring (turbidity) | BB | BB | BB | BB |
| precipitation | | | | |
| Stability | BB | BB | BB | BB |
| | Ex. (5-21) | Ex. (5-22) | Ex. (5-23) | Ex. (5-24) |
| Coloring (turbidity) | BB | BB | BB | BB |
| precipitation | | | | |
| Stability | BB | BB | BB | BB |
| | Ex. (5-25) | Ex. (5-26) | Ex. (5-27) | Ex. (5-28) |
| Coloring (turbidity) | BB | BB | BB | BB |
| precipitation | | | | |
| Stability | CC | CC | CC | CC |
| | Ex. (5-29) | Ex. (5-30) | Ex. (5-31) | Ex. (5-32) |
| Coloring (turbidity) | BB | BB | BB | BB |
| precipitation | | | | |
| Stability | CC | CC | CC | CC |
| | Ex. (5-33) | Ex. (5-34) | Ex. (5-35) | Ex. (5-36) |
| Coloring (turbidity) | BB | BB | BB | BB |
| precipitation | | | | |
| Stability | BB | BB | BB | BB |
| | Ex. (5-37) | Ex. (5-38) | Ex. (5-39) | Ex. (5-40) |
| Coloring (turbidity) | BB | BB | BB | BB |
| precipitation | | | | |
| Stability | BB | BB | BB | BB |
| | Ex. (5-41) | Ex. (5-42) | Ex. (5-43) | Ex. (5-44) |
| Coloring (turbidity) | BB | BB | BB | BB |
| precipitation | | | | |
| Stability | CC | CC | CC | CC |
| | Ex. (5-45) | Ex. (5-46) | Ex. (5-47) | Ex. (5-48) |
| Coloring (turbidity) | BB | BB | BB | BB |
| precipitation | | | | |
| Stability | CC | CC | CC | CC |

### Comparative Examples (C1-1) to (C1-4)

The components (i) to (ix) shown in Table 16 were homogeneously stirred and blended in accordance with a blending ratio shown in Table 16 and thereby dissolved to obtain a lotion.

The resulting lotion was placed in a closed container and allowed to stand still at 40° C. for 1 month. Then, occurrence of coloring (turbidity) and precipitation and a residue of the tocopherol glycine esters (A) were examined. The results are set forth in Table 18.

### Comparative Examples (C1-5) to (C1-48)

A lotion was prepared in the same manner as in Comparative Examples (C1-1) to (C1-4), except that the component (A) and the component (B) were changed in accordance with a combination shown in Table 17. The lotion was evaluated in the same manner as described above. The results are set forth in Table 18.

TABLE 16

| | | | Comp. | Comp. | Comp. | Comp. |
|---|---|---|---|---|---|---|
| | | | Ex. | Ex. | Ex. | Ex. |
| Component (% by mass) | | | (C1-1) | (C1-2) | (C1-3) | (C1-4) |
| (i) | (A) Tocopherol | A1 | 0.20 | 0.20 | 0.20 | 0.20 |
| | glycine esters | | | | | |
| (ii) | (B) Ascorbic | B1 | 3.00 | — | — | — |
| | acid-2-phosphoric | B2 | — | 3.00 | — | — |
| | acid esters | B3 | — | — | 3.00 | — |
| | | B4 | — | — | — | 3.00 |

| | | | | | |
|---|---|---|---|---|---|
| (iii) | Surfactin Na | 0.01 | 0.01 | 0.01 | 0.01 |
| (iv) | 1,3-Butylene glycol | 8.00 | 8.00 | 8.00 | 8.00 |
| (v) | Trehalose | 0.05 | 0.05 | 0.05 | 0.05 |
| (vi) | Citric acid | 0.20 | 0.20 | 0.20 | 0.20 |
| (vii) | Sodium citrate | 0.20 | 0.20 | 0.20 | 0.20 |
| (viii) | Methyl para- | 0.10 | 0.10 | 0.10 | 0.10 |
| | hydroxybenzoate | | | | |
| (ix) | Purified water | 88.24 | 88.24 | 88.24 | 88.24 |

TABLE 17

| | | (B) Ascorbic acid-2-phosphoric | | | |
|---|---|---|---|---|---|
| | | acid esters | | | |
| | | B1 | B2 | B3 | B4 |
| (A) | A1 | Comp. Ex. (C1-1) | Comp. Ex. (C1-2) | Comp. Ex. (C1-3) | Comp. Ex. (C1-4) |
| Tocopherol | A2 | Comp. Ex. (C1-5) | Comp. Ex. (C1-6) | Comp. Ex. (C1-7) | Comp. Ex. (C1-8) |
| glycine | A3 | Comp. Ex. (C1-9) | Comp. Ex. (C1-10) | Comp. Ex. (C1-11) | Comp. Ex. (C1-12) |
| esters | A4 | Comp. Ex. (C1-13) | Comp. Ex. (C1-14) | Comp. Ex. (C1-15) | Comp. Ex. (C1-16) |
| | A5 | Comp. Ex. (C1-17) | Comp. Ex. (C1-18) | Comp. Ex. (C1-19) | Comp. Ex. (C1-20) |
| | A6 | Comp. Ex. (C1-21) | Comp. Ex. (C1-22) | Comp. Ex. (C1-23) | Comp. Ex. (C1-24) |
| | A7 | Comp. Ex. (C1-25) | Comp. Ex. (C1-26) | Comp. Ex. (C1-27) | Comp. Ex. (C1-28) |
| | A8 | Comp. Ex. (C1-29) | Comp. Ex. (C1-30) | Comp. Ex. (C1-31) | Comp. Ex. (C1-32) |
| | A9 | Comp. Ex. (C1-33) | Comp. Ex. (C1-34) | Comp. Ex. (C1-35) | Comp. Ex. (C1-36) |
| | A10 | Comp. Ex. (C1-37) | Comp. Ex. (C1-38) | Comp. Ex. (C1-39) | Comp. Ex. (C1-40) |
| | A11 | Comp. Ex. (C1-41) | Comp. Ex. (C1-42) | Comp. Ex. (C1-43) | Comp. Ex. (C1-44) |
| | A12 | Comp. Ex. (C1-45) | Comp. Ex. (C1-46) | Comp. Ex. (C1-47) | Comp. Ex. (C1-48) |

TABLE 18

| | Comp. Ex. | Comp. Ex. | Comp. Ex. | Comp. Ex. |
|---|---|---|---|---|
| | (C1-1) | (C1-2) | (C1-3) | (C1-4) |
| Coloring (turbidity) | CC | CC | CC | CC |
| precipitation | | | | |
| Stability | CC | CC | CC | CC |
| | Comp. Ex. | Comp. Ex. | Comp. Ex. | Comp. Ex. |
| | (C1-5) | (C1-6) | (C1-7) | (C1-8) |
| Coloring (turbidity) | CC | CC | CC | CC |
| precipitation | | | | |
| Stability | CC | CC | CC | CC |
| | Comp. Ex. | Comp. Ex. | Comp. Ex. | Comp. Ex. |
| | (C1-9) | (C1-10) | (C1-11) | (C1-12) |
| Coloring (turbidity) | DD | DD | DD | DD |
| precipitation | | | | |
| Stability | DD | DD | DD | DD |
| | Comp. Ex. | Comp. Ex. | Comp. Ex. | Comp. Ex. |
| | (C1-13) | (C1-14) | (C1-15) | (C1-16) |
| Coloring (turbidity) | DD | DD | DD | DD |
| precipitation | | | | |
| Stability | DD | DD | DD | DD |
| | Comp. Ex. | Comp. Ex. | Comp. Ex. | Comp. Ex. |
| | (C1-17) | (C1-18) | (C1-19) | (C1-20) |
| Coloring (turbidity) | CC | CC | CC | CC |
| precipitation | | | | |
| Stability | CC | CC | CC | CC |
| | Comp. Ex. | Comp. Ex. | Comp. Ex. | Comp. Ex. |
| | (C1-21) | (C1-22) | (C1-23) | (C1-24) |
| Coloring (turbidity) | CC | CC | CC | CC |
| precipitation | | | | |
| Stability | CC | CC | CC | CC |
| | Comp. Ex. | Comp. Ex. | Comp. Ex. | Comp. Ex. |
| | (C1-25) | (C1-26) | (C1-27) | (C1-28) |
| Coloring (turbidity) | DD | DD | DD | DD |
| precipitation | | | | |
| Stability | DD | DD | DD | DD |
| | Comp. Ex. | Comp. Ex. | Comp. Ex. | Comp. Ex. |
| | (C1-29) | (C1-30) | (C1-31) | (C1-32) |
| Coloring (turbidity) | DD | DD | DD | DD |
| precipitation | | | | |
| Stability | DD | DD | DD | DD |
| | Comp. Ex. | Comp. Ex. | Comp. Ex. | Comp. Ex. |
| | (C1-33) | (C1-34) | (C1-35) | (C1-36) |
| Coloring (turbidity) | CC | CC | CC | CC |
| precipitation | | | | |
| Stability | CC | CC | CC | CC |
| | Comp. Ex. | Comp. Ex. | Comp. Ex. | Comp. Ex. |
| | (C1-37) | (C1-38) | (C1-39) | (C1-40) |
| Coloring (turbidity) | CC | CC | CC | CC |
| precipitation | | | | |
| Stability | CC | CC | CC | CC |
| | Comp. Ex. | Comp. Ex. | Comp. Ex. | Comp. Ex. |
| | (C1-41) | (C1-42) | (C1-43) | (C1-44) |
| Coloring (turbidity) | DD | DD | DD | DD |
| precipitation | | | | |
| Stability | DD | DD | DD | DD |
| | Comp. Ex. | Comp. Ex. | Comp. Ex. | Comp. Ex. |
| | (C1-45) | (C1-46) | (C1-47) | (C1-48) |
| Coloring (turbidity) | DD | DD | DD | DD |
| precipitation | | | | |
| Stability | DD | DD | DD | DD |

### Comparative Examples (C2-1) to (C2-4)

The components (i) to (ix) shown in Table 19 were homogeneously stirred and blended in accordance with a blending ratio shown in Table 19 and thereby dissolved to obtain a lotion.

The resulting lotion was placed in a closed container and allowed to stand still at 40° C. for 1 month. Then, occurrence of coloring (turbidity) and precipitation and a residue of the tocopherol glycine esters (A) were examined. The results are set forth in Table 21.

### Comparative Examples (C2-5) to (C2-48)

A lotion was prepared in the same manner as in Comparative Examples (C2-1) to (C2-4), except that the component (A) and the component (B) were changed in accordance with a combination shown in Table 20. The lotion was evaluated in the same manner as described above. The results are set forth in Table 21.

TABLE 19

| | | | Comp. | Comp. | Comp. | Comp. |
|---|---|---|---|---|---|---|
| | | | Ex. | Ex. | Ex. | Ex. |
| Component (% by mass) | | | (C2-1) | (C2-2) | (C2-3) | (C2-4) |
| (i) | (A) Tocopherol | A1 | 1.00 | 1.00 | 1.00 | 1.00 |
| | glycine esters | | | | | |
| (ii) | (B) Ascorbic | B1 | 1.00 | — | — | — |
| | acid-2-phosphoric | B2 | — | 1.00 | — | — |
| | acid esters | B3 | — | — | 1.00 | — |
| | | B4 | — | — | — | 1.00 |

| | | | | | |
|---|---|---|---|---|---|
| (iii) | Surfactin Na | 0.01 | 0.01 | 0.01 | 0.01 |
| (iv) | 1,3-Butylene glycol | 8.00 | 8.00 | 8.00 | 8.00 |
| (v) | Trehalose | 0.05 | 0.05 | 0.05 | 0.05 |
| (vi) | Citric acid | 0.20 | 0.20 | 0.20 | 0.20 |
| (vii) | Sodium citrate | 0.20 | 0.20 | 0.20 | 0.20 |
| (viii) | Methyl para- | 0.10 | 0.10 | 0.10 | 0.10 |
| | hydroxybenzoate | | | | |
| (ix) | Purified water | 89.44 | 89.44 | 89.44 | 89.44 |

TABLE 20

| | | (B) Ascorbic acid-2-phosphoric | | | |
|---|---|---|---|---|---|
| | | acid esters | | | |
| | | B1 | B2 | B3 | B4 |
| (A) | A1 | Comp. Ex. (C2-1) | Comp. Ex. (C2-2) | Comp. Ex. (C2-3) | Comp. Ex. (C2-4) |
| Tocopherol | A2 | Comp. Ex. (C2-5) | Comp. Ex. (C2-6) | Comp. Ex. (C2-7) | Comp. Ex. (C2-8) |
| glycine | A3 | Comp. Ex. (C2-9) | Comp. Ex. (C2-10) | Comp. Ex. (C2-11) | Comp. Ex. (C2-12) |
| esters | A4 | Comp. Ex. (C2-13) | Comp. Ex. (C2-14) | Comp. Ex. (C2-15) | Comp. Ex. (C2-16) |
| | A5 | Comp. Ex. (C2-17) | Comp. Ex. (C2-18) | Comp. Ex. (C2-19) | Comp. Ex. (C2-20) |
| | A6 | Comp. Ex. (C2-21) | Comp. Ex. (C2-22) | Comp. Ex. (C2-23) | Comp. Ex. (C2-24) |
| | A7 | Comp. Ex. (C2-25) | Comp. Ex. (C2-26) | Comp. Ex. (C2-27) | Comp. Ex. (C2-28) |
| | A8 | Comp. Ex. (C2-29) | Comp. Ex. (C2-30) | Comp. Ex. (C2-31) | Comp. Ex. (C2-32) |
| | A9 | Comp. Ex. (C2-33) | Comp. Ex. (C2-34) | Comp. Ex. (C2-35) | Comp. Ex. (C2-36) |
| | A10 | Comp. Ex. (C2-37) | Comp. Ex. (C2-38) | Comp. Ex. (C2-39) | Comp. Ex. (C2-40) |
| | A11 | Comp. Ex. (C2-41) | Comp. Ex. (C2-42) | Comp. Ex. (C2-43) | Comp. Ex. (C2-44) |
| | A12 | Comp. Ex. (C2-45) | Comp. Ex. (C2-46) | Comp. Ex. (C2-47) | Comp. Ex. (C2-48) |

TABLE 21

| | Comp. Ex. (C2-1) | Comp. Ex. (C2-2) | Comp. Ex. (C2-3) | Comp. Ex. (C2-4) |
|---|---|---|---|---|
| Coloring | DD | DD | DD | DD |
| (turbidity) | | | | |
| precipitation | | | | |
| Stability | DD | DD | DD | DD |
| | Comp. Ex. (C2-5) | Comp. Ex. (C2-6) | Comp. Ex. (C2-7) | Comp. Ex. (C2-8) |
| Coloring | DD | DD | DD | DD |
| (turbidity) | | | | |
| precipitation | | | | |
| Stability | DD | DD | DD | DD |
| | Comp. Ex. (C2-9) | Comp. Ex. (C2-10) | Comp. Ex. (C2-11) | Comp. Ex. (C2-12) |
| Coloring | DD | DD | DD | DD |
| (turbidity) | | | | |
| precipitation | | | | |
| Stability | DD | DD | DD | DD |
| | Comp. Ex. (C2-13) | Comp. Ex. (C2-14) | Comp. Ex. (C2-15) | Comp. Ex. (C2-16) |
| Coloring | DD | DD | DD | DD |
| (turbidity) | | | | |
| precipitation | | | | |
| Stability | DD | DD | DD | DD |
| | Comp. Ex. (C2-17) | Comp. Ex. (C2-18) | Comp. Ex. (C2-19) | Comp. Ex. (C2-20) |
| Coloring | DD | DD | DD | DD |
| (turbidity) | | | | |
| precipitation | | | | |
| Stability | DD | DD | DD | DD |
| | Comp. Ex. (C2-21) | Comp. Ex. (C2-22) | Comp. Ex. (C2-23) | Comp. Ex. (C2-24) |
| Coloring | DD | DD | DD | DD |
| (turbidity) | | | | |
| precipitation | | | | |
| Stability | DD | DD | DD | DD |
| | Comp. Ex. (C2-25) | Comp. Ex. (C2-26) | Comp. Ex. (C2-27) | Comp. Ex. (C2-28) |
| Coloring | DD | DD | DD | DD |
| (turbidity) | | | | |
| precipitation | | | | |
| Stability | DD | DD | DD | DD |
| | Comp. Ex. (C2-29) | Comp. Ex. (C2-30) | Comp. Ex. (C2-31) | Comp. Ex. (C2-32) |
| Coloring | DD | DD | DD | DD |
| (turbidity) | | | | |
| precipitation | | | | |
| Stability | DD | DD | DD | DD |
| | Comp. Ex. (C2-33) | Comp. Ex. (C2-34) | Comp. Ex. (C2-35) | Comp. Ex. (C2-36) |
| Coloring | DD | DD | DD | DD |
| (turbidity) | | | | |
| precipitation | | | | |
| Stability | DD | DD | DD | DD |
| | Comp. Ex. (C2-37) | Comp. Ex. (C2-38) | Comp. Ex. (C2-39) | Comp. Ex. (C2-40) |
| Coloring | DD | DD | DD | DD |
| (turbidity) | | | | |
| precipitation | | | | |
| Stability | DD | DD | DD | DD |
| | Comp. Ex. (C2-41) | Comp. Ex. (C2-42) | Comp. Ex. (C2-43) | Comp. Ex. (C2-44) |
| Coloring | DD | DD | DD | DD |
| (turbidity) | | | | |
| precipitation | | | | |
| Stability | DD | DD | DD | DD |
| | Comp. Ex. (C2-45) | Comp. Ex. (C2-46) | Comp. Ex. (C2-47) | Comp. Ex. (C2-48) |
| Coloring | DD | DD | DD | DD |
| (turbidity) | | | | |
| precipitation | | | | |
| Stability | DD | DD | DD | DD |

### Examples (6-1) to (6-4)

The components (i) to (vi) and the components (vii) to (xv) shown in Table 22 were each blended in accordance with a blending ratio shown in Table 22 and dissolved by heating at 80° C. to prepare a composition (I) and a composition (II). The composition (I) was slowly added to the composition (II) with stirring and thereby emulsified. The resulting liquid was cooled with stirring. At a temperature of 40 to 35° C., stirring was stopped, and the liquid was allowed to stand to prepare an emulsion.

The resulting emulsion was placed in a closed container and allowed to stand still at 40° C. for 1 month. Then, a residue of the tocopherol glycine esters (A) was examined. The results are set forth in Table 24.

### Examples (6-5) to (6-48)

An emulsion was prepared in the same manner as in Examples (6-1) to (6-4), except that the component (A) and the component (B) were changed in accordance with a combination shown in Table 23. The emulsion was evaluated in the same manner as described above. The results are set forth in Table 24.

TABLE 22

| | | | | Ex. | Ex. | Ex. | Ex. |
|---|---|---|---|---|---|---|---|
| Component (% by mass) | | | | (6-1) | (6-2) | (6-3) | (6-4) |
| (I) | (i) | (A) Tocopherol | A1 | 0.20 | 0.20 | 0.20 | 0.20 |
| | | glycine esters | | | | | |
| | (ii) | (B) Ascorbic | B1 | 3.00 | — | — | — |
| | | acid-2-phosphoric | B2 | — | 3.00 | — | — |
| | | acid esters | B3 | — | — | 3.00 | — |
| | | | B4 | — | — | — | 3.00 |

| | | | | | | |
|---|---|---|---|---|---|---|
| | (iii) | Surfactin Na | 1.00 | 1.00 | 1.00 | 1.00 |
| | (iv) | 1,3-Butylene glycol | 7.00 | 7.00 | 7.00 | 7.00 |
| | (v) | Xanthane gum | 0.10 | 0.10 | 0.10 | 0.10 |
| | (vi) | Purified water | 72.10 | 72.10 | 72.10 | 72.10 |
| (II) | (vii) | POE (20) sorbitan | 2.50 | 2.50 | 2.50 | 2.50 |
| | | monostearate | | | | |
| | (viii) | Lipophilic glyceryl | 1.00 | 1.00 | 1.00 | 1.00 |
| | | monostearate | | | | |
| | (ix) | Stearic acid | 0.50 | 0.50 | 0.50 | 0.50 |
| | (x) | Behenyl alcohol | 1.50 | 1.50 | 1.50 | 1.50 |
| | (xi) | Cetyl palmitate | 0.50 | 0.50 | 0.50 | 0.50 |
| | (xii) | Squalane | 5.00 | 5.00 | 5.00 | 5.00 |
| | (xiii) | Cetyl 2-ethylhexanoate | 5.00 | 5.00 | 5.00 | 5.00 |
| | (xiv) | Methyl polysiloxane | 0.50 | 0.50 | 0.50 | 0.50 |
| | | (100 cSt) | | | | |
| | (xv) | Methyl para- | 0.10 | 0.10 | 0.10 | 0.10 |
| | | hydroxybenzoate | | | | |

TABLE 23

| | | (B) Ascorbic acid-2-phosphoric | | | |
|---|---|---|---|---|---|
| | | acid esters | | | |
| | | B1 | B2 | B3 | B4 |
| (A) | A1 | Ex. (6-1) | Ex. (6-2) | Ex. (6-3) | Ex. (6-4) |
| Tocopherol | A2 | Ex. (6-5) | Ex. (6-6) | Ex. (6-7) | Ex. (6-8) |
| glycine | A3 | Ex. (6-9) | Ex. (6-10) | Ex. (6-11) | Ex. (6-12) |
| esters | A4 | Ex. (6-13) | Ex. (6-14) | Ex. (6-15) | Ex. (6-16) |
| | A5 | Ex. (6-17) | Ex. (6-18) | Ex. (6-19) | Ex. (6-20) |
| | A6 | Ex. (6-21) | Ex. (6-22) | Ex. (6-23) | Ex. (6-24) |
| | A7 | Ex. (6-25) | Ex. (6-26) | Ex. (6-27) | Ex. (6-28) |
| | A8 | Ex. (6-29) | Ex. (6-30) | Ex. (6-31) | Ex. (6-32) |
| | A9 | Ex. (6-33) | Ex. (6-34) | Ex. (6-35) | Ex. (6-36) |
| | A10 | Ex. (6-37) | Ex. (6-38) | Ex. (6-39) | Ex. (6-40) |
| | A11 | Ex. (6-41) | Ex. (6-42) | Ex. (6-43) | Ex. (6-44) |
| | A12 | Ex. (6-45) | Ex. (6-46) | Ex. (6-47) | Ex. (6-48) |

TABLE 24

| | Ex. (6-1) | Ex. (6-2) | Ex. (6-3) | Ex. (6-4) |
|---|---|---|---|---|
| Stability | AA | AA | AA | AA |
| | Ex. (6-5) | Ex. (6-6) | Ex. (6-7) | Ex. (6-8) |
| Stability | AA | AA | AA | AA |
| | Ex. (6-9) | Ex. (6-10) | Ex. (6-11) | Ex. (6-12) |
| Stability | BB | BB | BB | BB |
| | Ex. (6-13) | Ex. (6-14) | Ex. (6-15) | Ex. (6-16) |
| Stability | BB | BB | BB | BB |
| | Ex. (6-17) | Ex. (6-18) | Ex. (6-19) | Ex. (6-20) |
| Stability | AA | AA | AA | AA |
| | Ex. (6-21) | Ex. (6-22) | Ex. (6-23) | Ex. (6-24) |
| Stability | AA | AA | AA | AA |
| | Ex. (6-25) | Ex. (6-26) | Ex. (6-27) | Ex. (6-28) |
| Stability | BB | BB | BB | BB |
| | Ex. (6-29) | Ex. (6-30) | Ex. (6-31) | Ex. (6-32) |
| Stability | BB | BB | BB | BB |
| | Ex. (6-33) | Ex. (6-34) | Ex. (6-35) | Ex. (6-36) |
| Stability | AA | AA | AA | AA |
| | Ex. (6-37) | Ex. (6-38) | Ex. (6-39) | Ex. (6-40) |
| Stability | AA | AA | AA | AA |
| | Ex. (6-41) | Ex. (6-42) | Ex. (6-43) | Ex. (6-44) |
| Stability | BB | BB | BB | BB |
| | Ex. (6-45) | Ex. (6-46) | Ex. (6-47) | Ex. (6-48) |
| Stability | BB | BB | BB | BB |

### Examples (7-1) to (7-4)

The components (i) to (vi) and the components (vii) to (xv) shown in Table 25 were each blended in accordance with a blending ratio shown in Table 25 and dissolved by heating at 80° C. to prepare a composition (I) and a composition (II). The composition (I) was slowly added to the composition (II) with stirring and thereby emulsified. The resulting liquid was cooled with stirring. At a temperature of 40 to 35° C., stirring was stopped, and the liquid was allowed to stand to prepare an emulsion.

The resulting emulsion was placed in a closed container and allowed to stand still at 40° C. for 1 month. Then, a residue of the tocopherol glycine esters (A) was examined. The results are set forth in Table 27.

### Examples (7-5) to (7-48)

An emulsion was prepared in the same manner as in Examples (7-1) to (7-4), except that the component (A) and the component (B) were changed in accordance with a combination shown in Table 26. The emulsion was evaluated in the same manner as described above. The results are set forth in Table 27.

TABLE 25

| | | | | Ex. | Ex. | Ex. | Ex. |
|---|---|---|---|---|---|---|---|
| Component (% by mass) | | | | (7-1) | (7-2) | (7-3) | (7-4) |
| (I) | (i) | (A) Tocopherol | A1 | 1.00 | 1.00 | 1.00 | 1.00 |
| | | glycine esters | | | | | |
| | (ii) | (B) Ascorbic | B1 | 9.00 | — | — | — |
| | | acid-2-phosphoric | B2 | — | 9.00 | — | — |
| | | acid esters | B3 | — | — | 9.00 | — |
| | | | B4 | — | — | — | 9.00 |

| | | | | | | |
|---|---|---|---|---|---|---|
| | (iii) | Surfactin Na | 1.00 | 1.00 | 1.00 | 1.00 |
| | (iv) | 1,3-Butylene glycol | 7.00 | 7.00 | 7.00 | 7.00 |
| | (v) | Xanthane gum | 0.10 | 0.10 | 0.10 | 0.10 |
| | (vi) | Purified water | 65.30 | 65.30 | 65.30 | 65.30 |
| (II) | (vii) | POE (20) sorbitan | 2.50 | 2.50 | 2.50 | 2.50 |
| | | monostearate | | | | |
| | (viii) | Lipophilic glyceryl | 1.00 | 1.00 | 1.00 | 1.00 |
| | | monostearate | | | | |
| | (ix) | Stearic acid | 0.50 | 0.50 | 0.50 | 0.50 |
| | (x) | Behenyl alcohol | 1.50 | 1.50 | 1.50 | 1.50 |
| | (xi) | Cetyl palmitate | 0.50 | 0.50 | 0.50 | 0.50 |
| | (xii) | Squalane | 5.00 | 5.00 | 5.00 | 5.00 |
| | (xiii) | Cetyl 2-ethylhexanoate | 5.00 | 5.00 | 5.00 | 5.00 |
| | (xiv) | Methyl polysiloxane | 0.50 | 0.50 | 0.50 | 0.50 |
| | | (100 cSt) | | | | |
| | (xv) | Methyl para- | 0.10 | 0.10 | 0.10 | 0.10 |
| | | hydroxybenzoate | | | | |

TABLE 26

| | | (B) Ascorbic acid-2-phosphoric | | | |
|---|---|---|---|---|---|
| | | acid esters | | | |
| | | B1 | B2 | B3 | B4 |
| (A) | A1 | Ex. (7-1) | Ex. (7-2) | Ex. (7-3) | Ex. (7-4) |
| Tocopherol | A2 | Ex. (7-5) | Ex. (7-6) | Ex. (7-7) | Ex. (7-8) |
| glycine | A3 | Ex. (7-9) | Ex. (7-10) | Ex. (7-11) | Ex. (7-12) |
| esters | A4 | Ex. (7-13) | Ex. (7-14) | Ex. (7-15) | Ex. (7-16) |
| | A5 | Ex. (7-17) | Ex. (7-18) | Ex. (7-19) | Ex. (7-20) |
| | A6 | Ex. (7-21) | Ex. (7-22) | Ex. (7-23) | Ex. (7-24) |
| | A7 | Ex. (7-25) | Ex. (7-26) | Ex. (7-27) | Ex. (7-28) |
| | A8 | Ex. (7-29) | Ex. (7-30) | Ex. (7-31) | Ex. (7-32) |
| | A9 | Ex. (7-33) | Ex. (7-34) | Ex. (7-35) | Ex. (7-36) |
| | A10 | Ex. (7-37) | Ex. (7-38) | Ex. (7-39) | Ex. (7-40) |
| | A11 | Ex. (7-41) | Ex. (7-42) | Ex. (7-43) | Ex. (7-44) |
| | A12 | Ex. (7-45) | Ex. (7-46) | Ex. (7-47) | Ex. (7-48) |

TABLE 27

| | Ex. (7-1) | Ex. (7-2) | Ex. (7-3) | Ex. (7-4) |
|---|---|---|---|---|
| Stability | BB | BB | BB | BB |
| | Ex. (7-5) | Ex. (7-6) | Ex. (7-7) | Ex. (7-8) |
| Stability | BB | BB | BB | BB |
| | Ex. (7-9) | Ex. (7-10) | Ex. (7-11) | Ex. (7-12) |
| Stability | CC | CC | CC | CC |
| | Ex. (7-13) | Ex. (7-14) | Ex. (7-15) | Ex. (7-16) |
| Stability | CC | CC | CC | CC |
| | Ex. (7-17) | Ex. (7-18) | Ex. (7-19) | Ex. (7-20) |
| Stability | BB | BB | BB | BB |
| | Ex. (7-21) | Ex. (7-22) | Ex. (7-23) | Ex. (7-24) |
| Stability | BB | BB | BB | BB |
| | Ex. (7-25) | Ex. (7-26) | Ex. (7-27) | Ex. (7-28) |
| Stability | CC | CC | CC | CC |
| | Ex. (7-29) | Ex. (7-30) | Ex. (7-31) | Ex. (7-32) |
| Stability | CC | CC | CC | CC |
| | Ex. (7-33) | Ex. (7-34) | Ex. (7-35) | Ex. (7-36) |
| Stability | BB | BB | BB | BB |
| | Ex. (7-37) | Ex. (7-38) | Ex. (7-39) | Ex. (7-40) |
| Stability | BB | BB | BB | BB |
| | Ex. (7-41) | Ex. (7-42) | Ex. (7-43) | Ex. (7-44) |
| Stability | CC | CC | CC | CC |
| | Ex. (7-45) | Ex. (7-46) | Ex. (7-47) | Ex. (7-48) |
| Stability | CC | CC | CC | CC |

### Examples (8-1) to (8-4)

The components (i) to (vi) and the components (vii) to (xv) shown in Table 28 were each blended in accordance with a blending ratio shown in Table 28 and dissolved by heating at 80° C. to prepare a composition (I) and a composition (II). The composition (I) was slowly added to the composition (II) with stirring and thereby emulsified. The resulting liquid was cooled with stirring. At a temperature of 40 to 35° C., stirring was stopped, and the liquid was allowed to stand to prepare an emulsion.

The resulting emulsion was placed in a closed container and allowed to stand still at 40° C. for 1 month. Then, a residue of the tocopherol glycine esters (A) was examined. The results are set forth in Table 30.

### Examples (8-5) to (8-48)

An emulsion was prepared in the same manner as in Examples (8-1) to (8-4), except that the component (A) and the component (B) were changed in accordance with a combination shown in Table 29. The emulsion was evaluated in the same manner as described above. The results are set forth in Table 30.

TABLE 28

| | | | | Ex. | Ex. | Ex. | Ex. |
|---|---|---|---|---|---|---|---|
| Component (% by mass) | | | | (8-1) | (8-2) | (8-3) | (8-4) |
| (I) | (i) | (A) Tocopherol | A1 | 1.00 | 1.00 | 1.00 | 1.00 |
| | | glycine esters | | | | | |
| | (ii) | (B) Ascorbic | B1 | 1.00 | — | — | — |
| | | acid-2-phosphoric | B2 | — | 1.00 | — | — |
| | | acid esters | B3 | — | — | 1.00 | — |
| | | | B4 | — | — | — | 1.00 |

| | | | | | | |
|---|---|---|---|---|---|---|
| | (iii) | Surfactin Na | 1.00 | 1.00 | 1.00 | 1.00 |
| | (iv) | 1,3-Butylene glycol | 7.00 | 7.00 | 7.00 | 7.00 |
| | (v) | Xanthane gum | 0.10 | 0.10 | 0.10 | 0.10 |
| | (vi) | Purified water | 73.30 | 73.30 | 73.30 | 73.30 |
| (II) | (vii) | POE (20) sorbitan | 2.50 | 2.50 | 2.50 | 2.50 |
| | | Monostearate | | | | |
| | (viii) | Lipophilic glyceryl | 1.00 | 1.00 | 1.00 | 1.00 |
| | | Monostearate | | | | |
| | (ix) | Stearic acid | 0.50 | 0.50 | 0.50 | 0.50 |
| | (x) | Behenyl alcohol | 1.50 | 1.50 | 1.50 | 1.50 |
| | (xi) | Cetyl palmitate | 0.50 | 0.50 | 0.50 | 0.50 |
| | (xii) | Squalane | 5.00 | 5.00 | 5.00 | 5.00 |
| | (xiii) | Cetyl 2- | 5.00 | 5.00 | 5.00 | 5.00 |
| | | ethylhexanoate | | | | |
| | (xiv) | Methyl polysiloxane | 0.50 | 0.50 | 0.50 | 0.50 |
| | | (100 cSt) | | | | |
| | (xv) | Methyl para- | 0.10 | 0.10 | 0.10 | 0.10 |
| | | hydroxybenzoate | | | | |

TABLE 29

| | | (B) Ascorbic acid-2-phosphoric | | | |
|---|---|---|---|---|---|
| | | acid esters | | | |
| | | B1 | B2 | B3 | B4 |
| (A) | A1 | Ex. (8-1) | Ex. (8-2) | Ex. (8-3) | Ex. (8-4) |
| Tocopherol | A2 | Ex. (8-5) | Ex. (8-6) | Ex. (8-7) | Ex. (8-8) |
| glycine | A3 | Ex. (8-9) | Ex. (8-10) | Ex. (8-11) | Ex. (8-12) |
| esters | A4 | Ex. (8-13) | Ex. (8-14) | Ex. (8-15) | Ex. (8-16) |
| | A5 | Ex. (8-17) | Ex. (8-18) | Ex. (8-19) | Ex. (8-20) |
| | A6 | Ex. (8-21) | Ex. (8-22) | Ex. (8-23) | Ex. (8-24) |
| | A7 | Ex. (8-25) | Ex. (8-26) | Ex. (8-27) | Ex. (8-28) |
| | A8 | Ex. (8-29) | Ex. (8-30) | Ex. (8-31) | Ex. (8-32) |
| | A9 | Ex. (8-33) | Ex. (8-34) | Ex. (8-35) | Ex. (8-36) |
| | A10 | Ex. (8-37) | Ex. (8-38) | Ex. (8-39) | Ex. (8-40) |
| | A11 | Ex. (8-41) | Ex. (8-42) | Ex. (8-43) | Ex. (8-44) |
| | A12 | Ex. (8-45) | Ex. (8-46) | Ex. (8-47) | Ex. (8-48) |

TABLE 30

| | Ex. (8-1) | Ex. (8-2) | Ex. (8-3) | Ex. (8-4) |
|---|---|---|---|---|
| Stability | AA | AA | AA | AA |
| | Ex. (8-5) | Ex. (8-6) | Ex. (8-7) | Ex. (8-8) |
| Stability | AA | AA | AA | AA |
| | Ex. (8-9) | Ex. (8-10) | Ex. (8-11) | Ex. (8-12) |
| Stability | BB | BB | BB | BB |
| | Ex. (8-13) | Ex. (8-14) | Ex. (8-15) | Ex. (8-16) |
| Stability | BB | BB | BB | BB |
| | Ex. (8-17) | Ex. (8-18) | Ex. (8-19) | Ex. (8-20) |
| Stability | AA | AA | AA | AA |
| | Ex. (8-21) | Ex. (8-22) | Ex. (8-23) | Ex. (8-24) |
| Stability | AA | AA | AA | AA |
| | Ex. (8-25) | Ex. (8-26) | Ex. (8-27) | Ex. (8-28) |
| Stability | BB | BB | BB | BB |
| | Ex. (8-29) | Ex. (8-30) | Ex. (8-31) | Ex. (8-32) |
| Stability | BB | BB | BB | BB |
| | Ex. (8-33) | Ex. (8-34) | Ex. (8-35) | Ex. (8-36) |
| Stability | AA | AA | AA | AA |
| | Ex. (8-37) | Ex. (8-38) | Ex. (8-39) | Ex. (8-40) |
| Stability | AA | AA | AA | AA |
| | Ex. (8-41) | Ex. (8-42) | Ex. (8-43) | Ex. (8-44) |
| Stability | BB | BB | BB | BB |
| | Ex. (8-45) | Ex. (8-46) | Ex. (8-47) | Ex. (8-48) |
| Stability | BB | BB | BB | BB |

### Examples (9-1) to (9-4)

The components (i) to (vi) and the components (vii) to (xv) shown in Table 31 were each blended in accordance with a blending ratio shown in Table 31 and dissolved by heating at 80° C. to prepare a composition (I) and a composition (II). The composition (I) was slowly added to the composition (II) with stirring and thereby emulsified. The resulting liquid was cooled with stirring. At a temperature of 40 to 35° C., stirring was stopped, and the liquid was allowed to stand to prepare an emulsion.

The resulting emulsion was placed in a closed container and allowed to stand still at 40° C. for 1 month. Then, a residue of the tocopherol glycine esters (A) was examined. The results are set forth in Table 33.

### Examples (9-5) to (9-48)

An emulsion was prepared in the same manner as in Examples (9-1) to (9-4), except that the component (A) and the component (B) were changed in accordance with a combination shown in Table 32. The emulsion was evaluated in the same manner as described above. The results are set forth in Table 33.

TABLE 31

| Component (% by mass) | | | | Ex. (9-1) | Ex. (9-2) | Ex. (9-3) | Ex. (9-4) |
|---|---|---|---|---|---|---|---|
| (I) | (i) | (A) Tocopherol | A1 | 0.20 | 0.20 | 0.20 | 0.20 |
| | | glycine esters | | | | | |
| | (ii) | (B) Ascorbic | B1 | 3.00 | — | — | — |
| | | acid-2-phosphoric | B2 | — | 3.00 | — | — |
| | | acid esters | B3 | — | — | 3.00 | — |
| | | | B4 | — | — | — | 3.00 |

| | | | | | | |
|---|---|---|---|---|---|---|
| | (iii) | Surfactin Na | 0.10 | 0.10 | 0.10 | 0.10 |
| | (iv) | 1,3-Butylene glycol | 7.00 | 7.00 | 7.00 | 7.00 |
| | (v) | Xanthane gum | 0.10 | 0.10 | 0.10 | 0.10 |
| | (vi) | Purified water | 73.00 | 73.00 | 73.00 | 73.00 |
| (II) | (vii) | POE (20) sorbitan | 2.50 | 2.50 | 2.50 | 2.50 |
| | | monostearate | | | | |
| | (viii) | Lipophilic glyceryl | 1.00 | 1.00 | 1.00 | 1.00 |
| | | monostearate | | | | |
| | (ix) | Stearic acid | 0.50 | 0.50 | 0.50 | 0.50 |
| | (x) | Behenyl alcohol | 1.50 | 1.50 | 1.50 | 1.50 |
| | (xi) | Cetyl palmitate | 0.50 | 0.50 | 0.50 | 0.50 |
| | (xii) | Squalane | 5.00 | 5.00 | 5.00 | 5.00 |
| | (xiii) | Cetyl 2-ethylhexanoate | 5.00 | 5.00 | 5.00 | 5.00 |
| | (xiv) | Methyl polysiloxane (100 cSt) | 0.50 | 0.50 | 0.50 | 0.50 |
| | (xv) | Methyl para-hydroxybenzoate | 0.10 | 0.10 | 0.10 | 0.10 |

TABLE 32

| | | (B) Ascorbic acid-2-phosphoric acid esters | | | |
|---|---|---|---|---|---|
| | | B1 | B2 | B3 | B4 |
| (A) | A1 | Ex. (9-1) | Ex. (9-2) | Ex. (9-3) | Ex. (9-4) |
| Tocopherol | A2 | Ex. (9-5) | Ex. (9-6) | Ex. (9-7) | Ex. (9-8) |
| glycine | A3 | Ex. (9-9) | Ex. (9-10) | Ex. (9-11) | Ex. (9-12) |
| esters | A4 | Ex. (9-13) | Ex. (9-14) | Ex. (9-15) | Ex. (9-16) |
| | A5 | Ex. (9-17) | Ex. (9-18) | Ex. (9-19) | Ex. (9-20) |
| | A6 | Ex. (9-21) | Ex. (9-22) | Ex. (9-23) | Ex. (9-24) |
| | A7 | Ex. (9-25) | Ex. (9-26) | Ex. (9-27) | Ex. (9-28) |
| | A8 | Ex. (9-29) | Ex. (9-30) | Ex. (9-31) | Ex. (9-32) |
| | A9 | Ex. (9-33) | Ex. (9-34) | Ex. (9-35) | Ex. (9-36) |
| | A10 | Ex. (9-37) | Ex. (9-38) | Ex. (9-39) | Ex. (9-40) |
| | A11 | Ex. (9-41) | Ex. (9-42) | Ex. (9-43) | Ex. (9-44) |
| | A12 | EX. (9-45) | Ex. (9-46) | Ex. (9-47) | Ex. (9-48) |

TABLE 33

| | Ex. (9-1) | Ex. (9-2) | Ex. (9-3) | Ex. (9-4) |
|---|---|---|---|---|
| Stability | BB | BB | BB | BB |
| | Ex. (9-5) | Ex. (9-6) | Ex. (9-7) | Ex. (9-8) |
| Stability | BB | BB | BB | BB |
| | Ex. (9-9) | Ex. (9-10) | Ex. (9-11) | Ex. (9-12) |
| Stability | CC | CC | CC | CC |
| | Ex. (9-13) | Ex. (9-14) | Ex. (9-15) | Ex. (9-16) |
| Stability | CC | CC | CC | CC |
| | Ex. (9-17) | Ex. (9-18) | Ex. (9-19) | Ex. (9-20) |
| Stability | BB | BB | BB | BB |
| | Ex. (9-21) | Ex. (9-22) | Ex. (9-23) | Ex. (9-24) |
| Stability | BB | BB | BB | BB |
| | Ex. (9-25) | Ex. (9-26) | Ex. (9-27) | Ex. (9-28) |
| Stability | CC | CC | CC | CC |
| | Ex. (9-29) | Ex. (9-30) | Ex. (9-31) | Ex. (9-32) |
| Stability | CC | CC | CC | CC |
| | Ex. (9-33) | Ex. (9-34) | Ex. (9-35) | Ex. (9-36) |
| Stability | BB | BB | BB | BB |
| | Ex. (9-37) | Ex. (9-38) | Ex. (9-39) | Ex. (9-40) |
| Stability | BB | BB | BB | BB |
| | Ex. (9-41) | Ex. (9-42) | Ex. (9-43) | Ex. (9-44) |
| Stability | CC | CC | CC | CC |
| | Ex. (9-45) | Ex. (9-46) | Ex. (9-47) | Ex. (9-48) |
| Stability | CC | CC | CC | CC |

### Examples (10-1) to (10-4)

The components (i) to (vi) and the components (vii) to (xv) shown in Table 34 were each blended in accordance with a blending ratio shown in Table 34 and dissolved by heating at 80° C. to prepare a composition (I) and a composition (II). The composition (I) was slowly added to the composition (II) with stirring and thereby emulsified. The resulting liquid was cooled with stirring. At a temperature of 40 to 35° C., stirring was stopped, and the liquid was allowed to stand to prepare an emulsion.

The resulting emulsion was placed in a closed container and allowed to stand still at 40° C. for 1 month. Then, a residue of the tocopherol glycine esters (A) was examined. The results are set forth in Table 36.

### Examples (10-5) to (10-48)

An emulsion was prepared in the same manner as in Examples (10-1) to (10-4), except that the component (A) and the component (B) were changed in accordance with a combination shown in Table 35. The emulsion was evaluated in the same manner as described above. The results are set forth in Table 36.

TABLE 34

| Component (% by mass) | | | | Ex. (10-1) | Ex. (10-2) | Ex. (10-3) | Ex. (10-4) |
|---|---|---|---|---|---|---|---|
| (I) | (i) | (A) Tocopherol | A1 | 1.00 | 1.00 | 1.00 | 1.00 |
| | | glycine esters | | | | | |
| | (ii) | (B) Ascorbic | B1 | 1.00 | — | — | — |
| | | acid-2-phosphoric | B2 | — | 1.00 | — | — |
| | | acid esters | B3 | — | — | 1.00 | — |
| | | | B4 | — | — | — | 1.00 |

| | | | | | | |
|---|---|---|---|---|---|---|
| | (iii) | Surfactin Na | 0.10 | 0.10 | 0.10 | 0.10 |
| | (iv) | 1,3-Butylene glycol | 7.00 | 7.00 | 7.00 | 7.00 |
| | (v) | Xanthane gum | 0.10 | 0.10 | 0.10 | 0.10 |
| | (vi) | Purified water | 74.20 | 74.20 | 74.20 | 74.20 |
| (II) | (vii) | POE (20) sorbitan | 2.50 | 2.50 | 2.50 | 2.50 |
| | | monostearate | | | | |
| | (viii) | Lipophilic glyceryl | 1.00 | 1.00 | 1.00 | 1.00 |
| | | monostearate | | | | |
| | (ix) | Stearic acid | 0.50 | 0.50 | 0.50 | 0.50 |
| | (x) | Behenyl alcohol | 1.50 | 1.50 | 1.50 | 1.50 |
| | (xi) | Cetyl palmitate | 0.50 | 0.50 | 0.50 | 0.50 |
| | (xii) | Squalane | 5.00 | 5.00 | 5.00 | 5.00 |
| | (xiii) | Cetyl 2-ethylhexanoate | 5.00 | 5.00 | 5.00 | 5.00 |
| | (xiv) | Methyl polysiloxane (100 cSt) | 0.50 | 0.50 | 0.50 | 0.50 |
| | (xv) | Methyl para-hydroxybenzoate | 0.10 | 0.10 | 0.10 | 0.10 |

TABLE 35

| | | (B) Ascorbic acid-2-phosphoric acid esters | | | |
|---|---|---|---|---|---|
| | | B1 | B2 | B3 | B4 |
| (A) | A1 | Ex. (10-1) | Ex. (10-2) | Ex. (10-3) | Ex. (10-4) |
| Tocoph- | A2 | Ex. (10-5) | Ex. (10-6) | Ex. (10-7) | Ex. (10-8) |
| erol | A3 | Ex. (10-9) | Ex. (10-10) | Ex. (10-11) | Ex. (10-12) |
| glycine | A4 | Ex. (10-13) | Ex. (10-14) | Ex. (10-15) | Ex. (10-16) |
| esters | A5 | Ex. (10-17) | Ex. (10-18) | Ex. (10-19) | Ex. (10-20) |
| | A6 | Ex. (10-21) | Ex. (10-22) | Ex. (10-23) | Ex. (10-24) |
| | A7 | Ex. (10-25) | Ex. (10-26) | Ex. (10-27) | Ex. (10-28) |
| | A8 | Ex. (10-29) | Ex. (10-30) | Ex. (10-31) | Ex. (10-32) |
| | A9 | Ex. (10-33) | Ex. (10-34) | Ex. (10-35) | Ex. (10-36) |
| | A10 | Ex. (10-37) | Ex. (10-38) | Ex. (10-39) | Ex. (10-40) |
| | A11 | Ex. (10-41) | Ex. (10-42) | Ex. (10-43) | Ex. (10-44) |
| | A12 | Ex. (10-45) | Ex. (10-46) | Ex. (10-47) | Ex. (10-48) |

TABLE 36

| | Ex. (10-1) | Ex. (10-2) | Ex. (10-3) | Ex. (10-4) |
|---|---|---|---|---|
| Stability | BB | BB | BB | BB |
| | Ex. (10-5) | Ex. (10-6) | Ex. (10-7) | Ex. (10-8) |
| Stability | BB | BB | BB | BB |
| | Ex. (10-9) | Ex. (10-10) | Ex. (10-11) | Ex. (10-12) |
| Stability | CC | CC | CC | CC |
| | Ex. (10-13) | Ex. (10-14) | Ex. (10-15) | Ex. (10-16) |
| Stability | CC | CC | CC | CC |
| | Ex. (10-17) | Ex. (10-18) | Ex. (10-19) | Ex. (10-20) |
| Stability | BB | BB | BB | BB |
| | Ex. (10-21) | Ex. (10-22) | Ex. (10-23) | Ex. (10-24) |
| Stability | BB | BB | BB | BB |
| | Ex. (10-25) | Ex. (10-26) | Ex. (10-27) | Ex. (10-28) |
| Stability | CC | CC | CC | CC |
| | Ex. (10-29) | Ex. (10-30) | Ex. (10-31) | Ex. (10-32) |
| Stability | CC | CC | CC | CC |
| | Ex. (10-33) | Ex. (10-34) | Ex. (10-35) | Ex. (10-36) |
| Stability | BB | BB | BB | BB |
| | Ex. (10-37) | Ex. (10-38) | Ex. (10-39) | Ex. (10-40) |
| Stability | BB | BB | BB | BB |
| | Ex. (10-41) | Ex. (10-42) | Ex. (10-43) | Ex. (10-44) |
| Stability | CC | CC | CC | CC |
| | Ex. (10-45) | Ex. (10-46) | Ex. (10-47) | Ex. (10-48) |
| Stability | CC | CC | CC | CC |

### Comparative Examples (C3-1) to (C3-4)

The components (i) to (vi) and the components (vii) to (xv) shown in Table 37 were each blended in accordance with a blending ratio shown in Table 37 and dissolved by heating at 80° C. to prepare a composition (I) and a composition (II). The composition (I) was slowly added to the composition (II) with stirring and thereby emulsified. The resulting liquid was cooled with stirring. At a temperature of 40 to 35° C., stirring was stopped, and the liquid was allowed to stand to prepare an emulsion.

The resulting emulsion was placed in a closed container and allowed to stand still at 40° C. for 1 month. Then, a residue of the tocopherol glycine esters (A) was examined. The results are set forth in Table 39.

### Comparative Examples (C3-5) to (C3-48)

An emulsion was prepared in the same manner as in Comparative Examples (C3-1) to (C3-4), except that the component (A) and the component (B) were changed in accordance with a combination shown in Table 38. The emulsion was evaluated in the same manner as described above. The results are set forth in Table 39.

TABLE 37

| | | | | Com. Ex. | Com. Ex. | Com. Ex. | Com. Ex. |
|---|---|---|---|---|---|---|---|
| Component (% by mass) | | | | (C3-1) | (C3-2) | (C3-3) | (C3-4) |
| (I) | (i) | (A) Tocopherol | A1 | 0.20 | 0.20 | 0.20 | 0.20 |
| | | glycine esters | | | | | |
| | (ii) | (B) Ascorbic | B1 | 3.00 | — | — | — |
| | | acid-2-phosphoric | B2 | — | 3.00 | — | — |
| | | acid esters | B3 | — | — | 3.00 | — |
| | | | B4 | — | — | — | 3.00 |

| | | | | | | |
|---|---|---|---|---|---|---|
| | (iii) | Surfactin Na | 0.01 | 0.01 | 0.01 | 0.01 |
| | (iv) | 1,3-Butylene glycol | 7.00 | 7.00 | 7.00 | 7.00 |
| | (v) | Xanthane gum | 0.10 | 0.10 | 0.10 | 0.10 |
| | (vi) | Purified water | 73.10 | 73.10 | 73.10 | 73.10 |
| (II) | (vii) | POE (20) sorbitan | 2.50 | 2.50 | 2.50 | 2.50 |
| | | monostearate | | | | |
| | (viii) | Lipophilic glyceryl | 1.00 | 1.00 | 1.00 | 1.00 |
| | | monostearate | | | | |
| | (ix) | Stearic acid | 0.50 | 0.50 | 0.50 | 0.50 |
| | (x) | Behenyl alcohol | 1.50 | 1.50 | 1.50 | 1.50 |
| | (xi) | Cetyl palmitate | 0.50 | 0.50 | 0.50 | 0.50 |
| | (xii) | Squalane | 5.00 | 5.00 | 5.00 | 5.00 |
| | (xiii) | Cetyl 2-ethylhexanoate | 5.00 | 5.00 | 5.00 | 5.00 |
| | (xiv) | Methyl polysiloxane (100 cSt) | 0.50 | 0.50 | 0.50 | 0.50 |
| | (xv) | Methyl para-hydroxybenzoate | 0.10 | 0.10 | 0.10 | 0.10 |

TABLE 38

| | | (B) Ascorbic acid-2-phosphoric acid esters | | | |
|---|---|---|---|---|---|
| | | B1 | B2 | B3 | B4 |
| (A) | A1 | Com. Ex. (C3-1) | Com. Ex. (C3-2) | Com. Ex. (C3-3) | Com. Ex. (C3-4) |
| Tocopherol | A2 | Com. Ex. (C3-5) | Com. Ex. (C3-6) | Com. Ex. (C3-7) | Com. Ex. (C3-8) |
| glycine | A3 | Com. Ex. (C3-9) | Com. Ex. (C3-10) | Com. Ex. (C3-11) | Com. Ex. (C3-12) |
| esters | A4 | Com. Ex. (C3-13) | Com. Ex. (C3-14) | Com. Ex. (C3-15) | Com. Ex. (C3-16) |
| | A5 | Com. Ex. (C3-17) | Com. Ex. (C3-18) | Com. Ex. (C3-19) | Com. Ex. (C3-20) |
| | A6 | Com. Ex. (C3-21) | Com. Ex. (C3-22) | Com. Ex. (C3-23) | Com. Ex. (C3-24) |
| | A7 | Com. Ex. (C3-25) | Com. Ex. (C3-26) | Com. Ex. (C3-27) | Com. Ex. (C3-28) |
| | A8 | Com. Ex. (C3-29) | Com. Ex. (C3-30) | Com. Ex. (C3-31) | Com. Ex. (C3-32) |
| | A9 | Com. Ex. (C3-33) | Com. Ex. (C3-34) | Com. Ex. (C3-35) | Com. Ex. (C3-36) |
| | A10 | Com. Ex. (C3-37) | Com. Ex. (C3-38) | Com. Ex. (C3-39) | Com. Ex. (C3-40) |
| | A11 | Com. Ex. (C3-41) | Com. Ex. (C3-42) | Com. Ex. (C3-43) | Com. Ex. (C3-44) |
| | A12 | Com. Ex. (C3-45) | Com. Ex. (C3-46) | Com. Ex. (C3-47) | Com. Ex. (C3-48) |

TABLE 39

| | Com. Ex. | Com. Ex. | Com. Ex. | Com. Ex. |
|---|---|---|---|---|
| | (C3-1) | (C3-2) | (C3-3) | (C3-4) |
| Stability | CC | CC | CC | CC |
| | Com. Ex. | Com. Ex. | Com. Ex. | Com. Ex. |
| | (C3-5) | (C3-6) | (C3-7) | (C3-8) |
| Stability | CC | CC | CC | CC |
| | Com. Ex. | Com. Ex. | Com. Ex. | Com. Ex. |
| | (C3-9) | (C3-10) | (C3-11) | (C3-12) |
| Stability | DD | DD | DD | DD |
| | Com. Ex. | Com. Ex. | Com. Ex. | Com. Ex. |
| | (C3-13) | (C3-14) | (C3-15) | (C3-16) |
| Stability | DD | DD | DD | DD |
| | Com. Ex. | Com. Ex. | Com. Ex. | Com. Ex. |
| | (C3-17) | (C3-18) | (C3-19) | (C3-20) |
| Stability | CC | CC | CC | CC |
| | Com. Ex. | Com. Ex. | Com. Ex. | Com. Ex. |
| | (C3-21) | (C3-22) | (C3-23) | (C3-24) |
| Stability | CC | CC | CC | CC |
| | Com. Ex. | Com. Ex. | Com. Ex. | Com. Ex. |
| | (C3-25) | (C3-26) | (C3-27) | (C3-28) |
| Stability | DD | DD | DD | DD |
| | Com. Ex. | Com. Ex. | Com. Ex. | Com. Ex. |
| | (C3-29) | (C3-30) | (C3-31) | (C3-32) |
| Stability | DD | DD | DD | DD |
| | Com. Ex. | Com. Ex. | Com. Ex. | Com. Ex. |
| | (C3-33) | (C3-34) | (C3-35) | (C3-36) |
| Stability | CC | CC | CC | CC |
| | Com. Ex. | Com. Ex. | Com. Ex. | Com. Ex. |
| | (C3-37) | (C3-38) | (C3-39) | (C3-40) |
| Stability | CC | CC | CC | CC |
| | Com. Ex. | Com. Ex. | Com. Ex. | Com. Ex. |
| | (C3-41) | (C3-42) | (C3-43) | (C3-44) |
| Stability | DD | DD | DD | DD |
| | Com. Ex. | Com. Ex. | Com. Ex. | Com. Ex. |
| | (C3-45) | (C3-46) | (C3-47) | (C3-48) |
| Stability | DD | DD | DD | DD |

### Comparative Examples (C4-1) to (C4-4)

The components (i) to (vi) and the components (vii) to (xv) shown in Table 40 were each blended in accordance with a blending ratio shown in Table 40 and dissolved by heating at 80° C. to prepare a composition (I) and a composition (II). The composition (I) was slowly added to the composition (II) with stirring and thereby emulsified. The resulting liquid was cooled with stirring. At a temperature of 40 to 35° C., stirring was stopped, and the liquid was allowed to stand to prepare an emulsion.

The resulting emulsion was placed in a closed container and allowed to stand still at 40° C. for 1 month. Then, a residue of the tocopherol glycine esters (A) was examined. The results are set forth in Table 42.

### Comparative Examples (C4-5) to (C4-48)

An emulsion was prepared in the same manner as in Comparative Examples (C4-1) to (C4-4), except that the component (A) and the component (B) were changed in accordance with a combination shown in Table 41. The emulsion was evaluated in the same manner as described above. The results are set forth in Table 42.

TABLE 40

| | | | | Com. | Com | Com. | Com. |
|---|---|---|---|---|---|---|---|
| | | | | Ex. | Ex. | Ex. | Ex. |
| Component (% by mass) | | | | (C4-1) | (C4-2) | (C4-3) | (C4-4) |
| (I) | (i) | (A) Tocopherol | A1 | 1.00 | 1.00 | 1.00 | 1.00 |
| | | glycine esters | | | | | |
| | (ii) | (B) Ascorbic | B1 | 1.00 | — | — | — |
| | | acid-2-phosphoric | B2 | — | 1.00 | — | — |
| | | acid esters | B3 | — | — | 1.00 | — |
| | | | B4 | — | — | — | 1.00 |

| | | | | | | |
|---|---|---|---|---|---|---|
| | (iii) | Surfactin Na | 0.01 | 0.01 | 0.01 | 0.01 |
| | (iv) | 1,3-Butylene glycol | 7.00 | 7.00 | 7.00 | 7.00 |
| | (v) | Xanthane gum | 0.10 | 0.10 | 0.10 | 0.10 |
| | (vi) | Purified water | 74.30 | 74.30 | 74.30 | 74.30 |
| (II) | (vii) | POE (20) sorbitan | 2.50 | 2.50 | 2.50 | 2.50 |
| | | monostearate | | | | |
| | (viii) | Lipophilic glyceryl | 1.00 | 1.00 | 1.00 | 1.00 |
| | | monostearate | | | | |
| | (ix) | Stearic acid | 0.50 | 0.50 | 0.50 | 0.50 |
| | (x) | Behenyl alcohol | 1.50 | 1.50 | 1.50 | 1.50 |
| | (xi) | Cetyl palmitate | 0.50 | 0.50 | 0.50 | 0.50 |
| | (xii) | Squalane | 5.00 | 5.00 | 5.00 | 5.00 |
| | (xiii) | Cetyl 2-ethylhexanoate | 5.00 | 5.00 | 5.00 | 5.00 |
| | (xiv) | Methyl polysiloxane | 0.50 | 0.50 | 0.50 | 0.50 |
| | | (100 cSt) | | | | |
| | (xv) | Methyl para- | 0.10 | 0.10 | 0.10 | 0.10 |
| | | hydroxybenzoate | | | | |

TABLE 41

| | | (B) Ascorbic acid-2-phosphoric acid esters | | | |
|---|---|---|---|---|---|
| | | B1 | B2 | B3 | B4 |
| (A) | A1 | Com. Ex. (C4-1) | Com. Ex. (C4-2) | Com. Ex. (C4-3) | Com. Ex. (C4-4) |
| Tocopherol | A2 | Com. Ex. (C4-5) | Com. Ex. (C4-6) | Com. Ex. (C4-7) | Com. Ex. (C4-8) |
| glycine | A3 | Com. Ex. (C4-9) | Com. Ex. (C4-10) | Com. Ex. (C4-11) | Com. EX. (C4-12) |
| esters | A4 | Com. Ex. (C4-13) | Com. Ex. (C4-14) | Com. Ex. (C4-15) | Com. Ex. (C4-16) |
| | A5 | Com. Ex. (C4-17) | Com. Ex. (C4-18) | Com. Ex. (C4-19) | Com. Ex. (C4-20) |
| | A6 | Com. Ex. (C4-21) | Com. Ex. (C4-22) | Com. Ex. (C4-23) | Com. Ex. (C4-24) |
| | A7 | Com. Ex. (C4-25) | Com. Ex. (C4-26) | Com. Ex. (C4-27) | Com. Ex. (C4-28) |
| | A8 | Com. Ex. (C4-29) | Com. Ex. (C4-30) | Com. Ex. (C4-31) | Com. Ex. (C4-32) |
| | A9 | Com. Ex. (C4-33) | Com. Ex. (C4-34) | Com. Ex. (C4-35) | Com. Ex. (C4-36) |
| | A10 | Com. Ex. (C4-37) | Com. Ex. (C4-38) | Com. Ex. (C4-39) | Com. Ex. (C4-40) |
| | A11 | Com. Ex. (C4-41) | Com. Ex. (C4-42) | Com. Ex. (C4-43) | Com. Ex. (C4-44) |
| | A12 | Com. Ex. (C4-45) | Com. Ex. (C4-46) | Com. Ex. (C4-47) | Com. Ex. (C4-48) |

TABLE 42

| | Com. Ex. | Com. Ex. | Com. Ex. | Com. Ex. |
|---|---|---|---|---|
| | (C4-1) | (C4-2) | (C4-3) | (C4-4) |
| Stability | CC | CC | CC | CC |
| | Com. Ex. | Com. Ex. | Com. Ex. | Com. Ex. |
| | (C4-5) | (C4-6) | (C4-7) | (C4-8) |
| Stability | CC | CC | CC | CC |
| | Com. Ex. | Com. Ex. | Com. Ex. | Com. Ex. |
| | (C4-9) | (C4-10) | (C4-11) | (C4-12) |
| Stability | DD | DD | DD | DD |
| | Com. Ex. | Com. Ex. | Com. Ex. | Com. Ex. |
| | (C4-13) | (C4-14) | (C4-15) | (C4-16) |
| Stability | DD | DD | DD | DD |
| | Com. Ex. | Com. Ex. | Com. Ex. | Com. Ex. |
| | (C4-17) | (C4-18) | (C4-19) | (C4-20) |
| Stability | CC | CC | CC | CC |
| | Com. Ex. | Com. Ex. | Com. Ex. | Com. Ex. |
| | (C4-21) | (C4-22) | (C4-23) | (C4-24) |
| Stability | CC | CC | CC | CC |
| | Com. Ex. | Com. Ex. | Com. Ex. | Com. Ex. |
| | (C4-25) | (C4-26) | (C4-27) | (C4-28) |
| Stability | DD | DD | DD | DD |
| | Com. Ex. | Com. Ex. | Com. Ex. | Com. Ex. |
| | (C4-29) | (C4-30) | (C4-31) | (C4-32) |
| Stability | DD | DD | DD | DD |
| | Com. Ex. | Com. Ex. | Com. Ex. | Com. Ex. |
| | (C4-33) | (C4-34) | (C4-35) | (C4-36) |
| Stability | CC | CC | CC | CC |
| | Com. Ex. | Com. Ex. | Com. Ex. | Com. Ex. |
| | (C4-37) | (C4-38) | (C4-39) | (C4-40) |
| Stability | CC | CC | CC | CC |
| | Com. Ex. | Com. Ex. | Com. Ex. | Com. Ex. |
| | (C4-41) | (C4-42) | (C4-43) | (C4-44) |
| Stability | DD | DD | DD | DD |
| | Com. Ex. | Com. Ex. | Com. Ex. | Com. Ex. |
| | (C4-45) | (C4-46) | (C4-47) | (C4-48) |
| Stability | DD | DD | DD | DD |

### INDUSTRIAL APPLICABILITY

The present invention can prepare an agent for skin external use having such properties that the stability of tocopherol glycine esters is enhanced, the occurrence of coloring (including turbidity) and/or precipitation caused with time can be prevented and the agent can effectively exhibite the efficiency for a long time, although it contains tocopherol glycine esters and ascorbic acids. Accordingly, the present invention is useful for preparation of agents for skin external use, particularly cosmetics.

1. An agent for skin external use, comprising:
(A) at least one substance selected from the group consisting of tocopherol glycine ester, a tocopherol glycine ester derivative and a salt thereof,
(B) at least one substance selected from the group consisting of ascorbic acid-2-phosphoric acid ester, an ascorbic acid-2-phosphoric acid ester derivative and a salt thereof, and
(C) an anionic surface active agent having a lipopeptide structure,
wherein the anionic surface active agent (C) having a lipopeptide structure is contained in an amount of 0.03 to 20% by mass.

2. The agent for skin external use as claimed in claim 1, wherein the component (A) has an α-tocopherol skeleton, ι-tocopherol skeleton or δ-tocopherol skeleton structure.

3. The agent for skin external use as claimed in claim 1, wherein the component (A) has a dl-α-tocopherol skeleton, d-α-tocopherol skeleton, d-γ-tocopherol skeleton or d-δ-tocopherol skeleton structure.

4. The agent for skin external use as claimed in claim 1 wherein the component (A) is an organic acid salt of a tocopherol glycine ester.

5. The agent for skin external use as claimed in claim 1 wherein the component (A) is an inorganic acid salt of a tocopherol glycine ester.

6. The agent for skin external use as claimed in claim 5 wherein the inorganic acid salt of a tocopherol glycine ester is a hydrochloride.

7. The agent for skin external use as claimed in of claim 1 wherein the component (A) is an organic acid salt of a tocophrol glycine ester derivative.

8. The agent for skin external use as claimed in of claim 1 wherein the component (A) is an inorganic acid salt of a tocophrol glycine ester derivative.

9. The agent for skin external use as claimed in claim 8 wherein the inorganic acid salt of a tocopherol glycine ester derivative is a hydrochloride.

10. The agent for skin external use as claimed in claim 1, wherein the tocopherol glycine ester derivative is a compound represented by the formula (1): 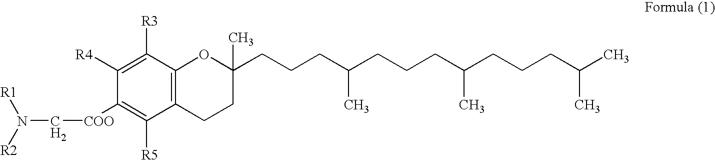
wherein R1 and R2 are the same or different and are each a lower alkyl group or a hydrogen atom with the proviso that they are not hydrogen atoms at the same time, and R3, R4 and R5 are the same or different and are each a hydrogen atom or a methyl group.

11. The agent for skin external use as claimed in claim 10, wherein the tocopherol glycine ester derivative is N,N-dimethylglycine ester of tocopherol or tocopherol sarcosine ester provided that in the formula (1), R1 and R2, which are simultaneously not hydrogens, are identically or differently a methyl group or hydrogen.

12. The agent for skin external use as claimed in claim 1, wherein the component (A) is contained in an amount of 0.1 to 10% by mass.

13. The agent for skin external use as claimed in claim 1, wherein the component (B) is a sodium salt of the ascorbic acid-2-phosphoric acid ester.

14. The agent for skin external use as claimed in claim 1, wherein the component (B) is a magnesium salt of the ascorbic acid-2-phosphoric acid ester.

15. The agent for skin external use as claimed in claim 1, wherein the ascorbic acid-2-phosphoric acid ester derivative is a compound represented by the formula (2): 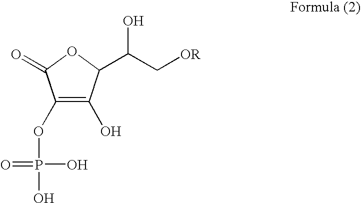
wherein R is an acyl group derived from a higher fatty acid.

16. The agent for skin external use as claimed in claim 1, wherein the component (B) is an ascorbic acid-2-phosphoric acid ester-6-palmitic acid ester sodium salt.

17. The agent for skin external use as claimed in claim 1, wherein the component (B) is an ascorbic acid-2-phosphoric acid ester-6-hexyldecanoic acid ester sodium salt.

18. The agent for skin external use as claimed in claim 1, wherein the component (B) is contained in an amount of 0.1 to 10% by mass.

19. The agent for skin external use as claimed in claim 1, wherein the component (C) is a surfactin and/or a salt thereof.

20. The agent for skin external use as claimed in claim 19, wherein the component (C) is a sodium salt of a surfactin.

21. The agent for skin external use as claimed in claim 19, wherein the surfactin contains one or more compounds represented by the formula (3): 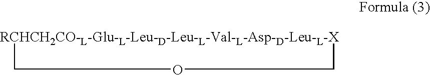
wherein X is an amino acid residue selected from the group consisting of leucine, isoleucine, valine, glycine, serine, alanine, threonine, asparagine, glutamine, aspartic acid, glutamic acid, lysine, arginine, cysteine, methionine, phenylalanine, tyrosine, tryptophan, histidine, proline, 4-hydroxyproline and homoserine, and R is a normal alkyl group of 8 to 14 carbon atoms, an isoalkyl group of 8 to 14 carbon atoms or an anteisoalkyl group of 8 to 14 carbon atoms.

22. The agent for skin external use as claimed in claim 21, wherein X in the formula (3) is leucine, isoleucine or valine.

23. The agent for skin external use as claimed in claim 1, which agent further comprises (D) water.

24. The agent for skin external use as claimed in claim 1 which agent is a cosmetic.
